(19) United States
(12) Patent Application Publication
(10) Pub. No.: US 2026/0108254 A1
(43) Pub. Date: Apr. 23, 2026

(54) ORTHOPEDIC SURGICAL GUIDE AND ASSOCIATED SYSTEMS AND METHOD AND FEELER

(71) Applicant: NOVASTEP, RENNES (FR)

(72) Inventors: Marek MURMANSKI, Rennes (FR); Chloé LAROCHE, Rennes (FR); Loïc GIROD, Goven (FR); Brian SCHAUER, Huntersville, NC (US)

(21) Appl. No.: 19/116,955

(22) PCT Filed: Jul. 13, 2023
(86) PCT No.: PCT/IB2023/000428

§ 371 Date: Mar. 28, 2025

Related U.S. Application Data
(60) Provisional application No. 63/411,862, filed on Sep. 30, 2022.

(51) Int. Cl. A61B 17/15; A61B 17/86

(52) U.S. Cl. CPC A61B 17/151; A61B 17/86

(57) ABSTRACT

An orthopedic surgical guide for a surgical operation to fuse two bone fragments, wherein the guide includes:—a frame configured to be removably attached to at least one of the two bone fragments,—a cutting window

### TECHNICAL FIELD

The present invention concerns the field of surgical guides. More particularly, the instant disclosure relates to a reusable guide for surgery by osteosynthesis of the cuneo-metatarsal joint, for example of the first ray, in particular in a minimally invasive or percutaneous approach.

### STATE OF THE ART

It is well-known that hallux valgus deformity results from cuneo-metatarsal hypermobility during a metatarsus varus. The metatarsus varus is accentuated during the weight-bearing phase. The first metatarsal then deviates inwards (varus), while the first phalanx, held by its joint capsule and the sesamoid strap, deviates outwards (valgus).

There are different surgical techniques to correct this deformity.

In the context of minimally invasive or percutaneous surgery for the correction of hallux valgus, one of the techniques commonly used is Lapidus arthrodesis, which includes fusing the cuneo-metatarsal joint by means of at least two compressive screws inserted crosswise after correction of the varus.

This technique is common, for example, in cases where the intermetatarsal angle (commonly known as M1-M2, M1 and M2 referring respectively to the first and second metatarsal bones) is greater than 20° together with instability of the cuneo-metatarsal joint, because it allows for large displacements of the bone(s). Its intention is to correct a metatarsus primus varus to correct a “functional” instability of the first ray. This procedure can also be performed when there is a risk of necrosis of the metatarsal head.

During the surgical procedure, the reduction of the first metatarsal and its stabilization is the most decisive and also the most complex step to perform. The extent of which the position and orientation of the metatarsal are correct will condition the bone cut and thus the anatomical congruence of the bone fragments during the placement of the two cross-shaped screws that will perform the osteosynthesis. This position will also ensure good biomechanical support of the foot.

For performing the cutting step, the practitioner may use several methods with either burs, curettes/gouges or an oscillating saw.

These preparation options are often performed freehand and in open approach, usually including an angular adjustment which then generates a specific correction once the two fragments, one of the cuneiform bone and the other of the metatarsal bone, are placed face to face. This is therefore subject to a high level of uncertainty at the time of preparation and usually requires a long surface adjustment phase.

For performing such adjustment to obtain such correction, the practitioner's hand will be used to correctly position the distal part of the metatarsal bone. In some cases, a distraction/compression forceps can be used. Osteosynthesis is then performed with pin-guided screws, most often in a cross-screw construct, but also alternatively with a combination of plates and osteosynthesis screws. Since the correction is multidirectional—it implies a reduction of the intermetatarsal angle, a supination of the metatarsal to align with the sesamoids, an elevation of the metatarsal to maintain a good distribution of support, and a compression on the cuneiform—stabilization of the correction is a critical point.

Usually, the steps of M1 reduction (reducing the M1-M2 angle by displacement of the first metatarsal bone), surface preparation and pin insertion are done under fluoroscopy. This results in numerous of X-ray images, which can be detrimental to the health of both patients and operators. In addition, the bone surfaces are not always congruent and the metatarsal is not correctly reduced in all directions, resulting in poor implant positioning, possible mechanical weakness of the system, and therefore failure of the operation.

In order to remedy these drawbacks, several systems have been developed to try to guide the reduction of the first metatarsal. This is the case, in particular, for the system described in U.S. Pat. No. 9,622,805 or the device described in US20210251670.

The system of U.S. Pat. No. 9,622,805 is based on two elements: a cutting guide and a reducer. The cutting guide offers two oscillating saw cutting zones for bone cuts. However, it offers only one possible angulation between the two cuts of the facing bones, and requires open surgery to perform the oscillating saw cuts through the cutting zones. One part of the reducer is positioned against the 2^{nd} metatarsal, thus requiring an additional incision. By turning a knob of the reducer, the inter-metatarsal angle is reduced and a certain supination occurs naturally. A pin is then applied to stabilize the unit in the desired position. The bearing on the bone of the 2^{nd} metatarsal accentuates the invasive nature of the technique. Moreover, this system does not allow the management of the elevation or lowering of the metatarsal, which remains a manual operation.

The system described in US20210251670 is similar to the system of U.S. Pat. No. 9,622,805 in terms of operation. It also uses an oscillating saw cutting guide and a reducer positioned on the 2^{nd} metatarsal. In addition to the system described above, it includes a lever that can be fixed to the distal part of the cutting guide in order to supinate the metatarsal. The cutting guide offers only one cutting option, and is thus symmetrical in order to be used on both left foot and right foot.

Both of these prior art systems have the disadvantage of requiring a large incision to make the saw cuts, as well as to position the future implants, which include plates and osteosynthesis screws. In addition, a second incision is required for the reducer to be positioned, which increases the invasiveness. Furthermore, these systems still require many manual operations from the operator.

### SUMMARY

Hence, one amongst the objects of the invention is to overcome at least one of these drawbacks by providing an orthopedic surgical guide, and associated systems and method, and feeler.

To this end, and in accordance with the invention, there is provided an orthopedic surgical guide for a surgical operation to fuse two bone fragments, wherein the guide includes:
a frame configured to be removably attached to at least one of the two bone fragments,
a cutting window.

The guide can include the following features, taken alone or according to any technically possible combination:
the window is configured for allowing the passing through of at least a part of a cutting tool for cutting at least one of the two bone fragments,
the window is configured to allow the insertion of at least a part of a feeler by relative displacement of the window with respect to the feeler so that the feeler guides the frame,
the frame is configured to be removably attached to a single of the bone fragments to be fused,
the guide further includes a correction unit,
the frame and correction unit are configured so that the correction unit can be removably attached to the frame,
the correction unit includes a paddle configured to be in contact with one of the two bone fragments,
the guide further includes a rotation unit,
the frame and rotation unit are configured so that the rotation unit can be removably attached to the frame,
the rotation unit has at least one pin opening, each being configured to allow passage of a pin,
the guide is configured to allow the displacement of one of the two bone fragments relative to the other of the two bone fragments, so as to allow at least two degrees in freedom,
the guide further includes an aiming unit,
the frame and aiming unit are configured so that the aiming unit can be removably attached to the frame,
the aiming unit includes at least one slide,
the aiming unit includes a first arm and a second arm, the first arm including a first housing configured for receiving a first barrel,
the guide further comprises the first barrel and the second barrel, such that, when the first housing receives the first barrel, the first barrel being configured to receive a first guiding pin and the second barrel being configured to receive a second guiding pin, so as to position the first guiding pin and the second guiding pin with respect to at least one of the bone fragments,
   the first guiding pin being configured, when positioned, to guide a first orthopedic implant, and
   the second guiding pin being configured, when positioned, to guide a second orthopedic implant.

In accordance with the invention, there is also provided an orthopedic surgical guide system for a surgical operation to fuse two bone fragments, wherein the system includes:
the guide, and
the first guiding pin and the second guiding pin, the first guiding pin being configured, when positioned, to guide a first orthopedic implant, and
the second guiding pin being configured, when positioned, to guide a second orthopedic implant.

The system can include the following features, taken alone or according to any technically possible combination:
the first orthopedic implant and the second orthopedic implant, wherein the first orthopedic implant is a screw and the second orthopedic implant is a screw,
a feeler to be inserted between the two bone fragments, the feeler being configured to cooperate with the guide,
the window has a guiding opening for inserting at least a part of a feeler through the guiding opening by relative displacement of the window with respect to the feeler so that the feeler guides the frame,
the feeler has a rib adapted to cooperate with the guiding opening of the window of the guide, and
the feeler has a first tapered end.

In accordance with the invention, there is also provided an orthopedic surgical guide system for a surgical operation to fuse two bone fragments, wherein the system includes:
the guide, and
a feeler to be inserted between the two bone fragments, the feeler being configured to cooperate with the guide.

The system can include the following features, taken alone or according to any technically possible combination:
the window has a guiding opening for inserting at least a part of a feeler through the guiding opening by relative displacement of the window with respect to the feeler so that the feeler guides the frame,
the feeler has a rib adapted to cooperate with the guiding opening of the window of the guide, and
the feeler has a first tapered end.

In accordance with the invention, there is also provided a feeler for use in a surgical operation to fuse two bone fragments, the feeler being configured to cooperate with an orthopedic surgical guide for the surgical operation to fuse two bone fragments.

The feeler can include the following features, taken alone or according to any technically possible combination:
the feeler has a first tapered end, and
the feeler has a rib adapted to cooperate with a guiding opening of a window of the guide.

In accordance with the invention, there is also provided a surgical method to fuse two bone fragments, implemented by the system, the method including:
performing cut(s) on the first and/or second bone fragment(s),
performing correction(s) of the position of at least one of the two bone fragments, for example the first bone fragment and/or second bone fragment.

### BRIEF DESCRIPTION OF THE DRAWINGS

Other advantages and features will appear better from the following description of several variants, provided as non-limiting examples, of the orthopedic surgical guide, and associated systems and method, and feeler in accordance with the invention, with reference to the appended drawings wherein:

FIG. 1a shows a perspective view of the guide and system according to an example of a first embodiment of the invention with a rotation unit, in position on a foot,

FIG. 1b shows a perspective view of the guide and system according to an example of a first embodiment of the invention with an aiming unit, in position on a foot,

FIG. 2 represents a perspective view of a feeler according to an example of a first embodiment of the invention,

FIG. 3 represents a perspective view of a cutting window of a guide and system according to an example of a first embodiment of the invention,

FIG. 4 represents a perspective view of a frame of a guide and system according to an example of a first embodiment of the invention,

FIG. 5 represents a perspective view of a guide and system with a correction unit according to an example of a first embodiment of the invention,

FIGS. 6a, 6b and 6c represent views of rotation unit of a guide and system with a correction unit according to an example of a first embodiment of the invention,

FIG. 7 represents a perspective view of a guide and system with an aiming unit according to an example of a first embodiment of the invention,

FIG. 8a represents a perspective view of a feeler according to an example of a first embodiment of the invention inserted in a foot joint,

FIG. 8b represents a first cut performed by means of a guide according to an example of a first embodiment of the invention cooperating with the feeler of FIG. 8a,

FIG. 9 represents a second cut performed by means of a guide according to an example of a first embodiment of the invention,

FIGS. 10 and 11 represent corrections by a correction unit and rotation unit of a guide according to an example of a first embodiment of the invention,

FIG. 12 to 23 represent steps of a surgical method performed by means of a guide and a system according to an example of a first embodiment of the invention

FIG. 24a shows a perspective view of the guide and system according to an example of a second embodiment of the invention with a rotation unit, in position on a foot,

FIG. 24b shows a perspective view of the guide and system according to an example of a second embodiment of the invention with an aiming unit, in position on a foot,

FIG. 25a represents a perspective view of a feeler according to an example of a second embodiment of the invention,

FIG. 25b represents a side view of the feeler of FIG. 25a,

FIG. 26 represents a perspective view of a cutting window of a guide and system according to an example of a second embodiment of the invention,

FIG. 27a represents a perspective view from a high angle of a frame of a guide and system according to an example of a second embodiment of the invention,

FIG. 27b represents a perspective view from a low angle of the frame of FIG. 27a,

FIG. 27c represents a cross-sectional view of the frame of FIG. 27a with the cutting window of FIG. 26 in a plane parallel to the plane defined by a cutting hole of the window, in particular the plane defined by axes X3 and Z3,

FIG. 28a represents a perspective view from a high angle of a guide and system with a correction unit according to an example of a second embodiment of the invention,

FIG. 28b represents a cross-sectional view of the guide and system of FIG. 28a in the plane defined by axes X5 and Z5,

FIG. 28c represents another cross-sectional view from the side of the guide and system of FIG. 28a in the plane defined by axes Y5 and Z5,

FIG. 29 represent a perspective view of a support subunit of a guide and system with a correction unit according to an example of a second embodiment of the invention,

FIG. 30a represents a perspective view of an aiming subunit of a guide and system according to an example of a second embodiment of the invention,

FIG. 30b represents a cross-sectional view of the aiming subunit of FIG. 30a in a plane defined by axes X81 and Z8,

FIG. 30c represents a view from above of the aiming subunit of FIG. 30a,

FIG. 30d represents a partial cross-sectional view of the aiming unit of FIG. 30a with a part of the cross-sectional view corresponding to a plane defined by axes X81 and Z8 and another part of the cross-sectional view corresponding to a plane including axis X82 and parallel to the plane defined by axes X81 and Z8,

FIG. 31 represent a side view of a locking wheel of the rotation unit of a guide and system with a correction unit according to an example of a second embodiment of the invention,

FIG. 32 represents a view of a guide and system with an aiming unit according to an example of a second embodiment of the invention,

FIG. 33 to 43 represent steps of a surgical method performed by means of a guide and a system according to an example of a second embodiment of the invention.

### DETAILED DESCRIPTION

In the following description, the same reference numerals refer to the same elements. The different views are not necessarily plotted to scale. Moreover, the surgical guide, system(s), feeler and method according to the invention are particularly suited to the fusion of the cuneo-metatarsal joint (Lapidus fusion), for example the fusion of the first cuneo-metatarsal joint. But it is quite obvious that it could be suited to the metatarsophalangeal joint (MTP) fusion, for example the first metatarsophalangeal joint (MTP) fusion, yet without departing from the scope of the invention.

By minimally invasive approach, it is meant surgery limiting the approach to a few centimeters and by percutaneous approach, surgery limiting the approach to a few millimeters.

### Guide

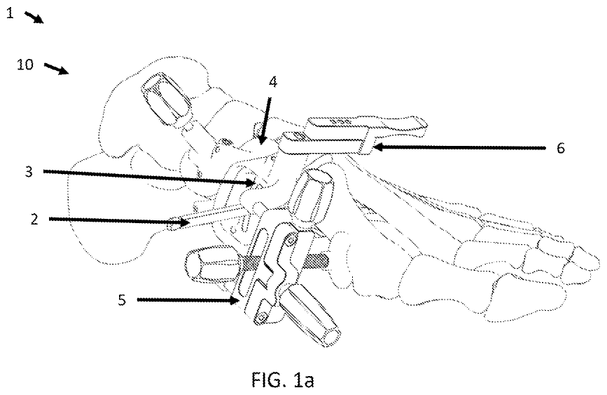

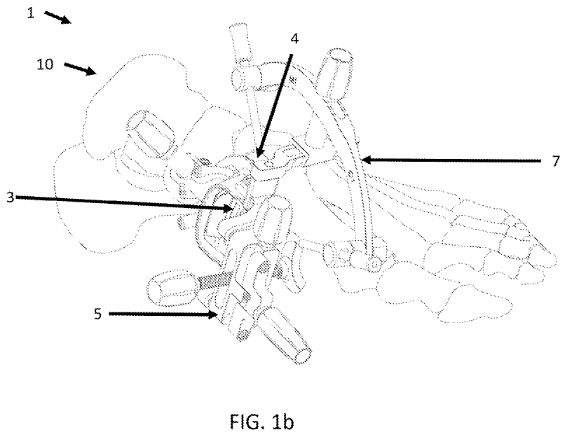

With reference to FIG. 1a-1b, 3 to 7, 24a-b, 26 to 31, a surgical guide 10, for example an orthopedic surgical guide 10 is described. The guide 10 is for example a reusable guide. The guide 10 is for example an orthopedic surgical guide for a surgical operation to fuse two bone fragments.

The guide 10 is for example an orthopedic surgical guide 10 for joint preparation and/or bone correction and/or implant guidance, in a surgical operation to fuse two bone fragments, for example a Lapidus procedure.

The guide 10 includes a frame 4 configured to be removably attached to at least one of the two bone fragments, for example foot bone, for example a cuneiform bone, for example the first cuneiform bone. The guide 10 includes a cutting window 3. The cutting window 3 can have at least one opening 3a and/or 3b, for example only one opening 3b.

By saying that an element can be removably attached, it is for example meant that an element can be attached and removed without the element being deteriorated and/or while keeping the element in a reusable state. By saying that an element is non-removably attached, it is for example meant that an element is attached and cannot be removed without the element being deteriorated and/or without putting the element in a non-reusable state.

The guide 10 can further include a correction unit 5. Alternatively or in combination, the guide 10 can further include a rotation unit 6, for example a correction and rotation unit including the correction unit 5 and the rotation unit 6. Alternatively or in combination, the guide can further include an aiming unit 7. The guide 10, including the correction unit 5 and/or rotation unit 6 forms for example a multidirectional reduction device. The guide 10, including the aiming unit 7, forms for example an anatomically adapted aiming device.

The described surgical guide, for example for Lapidus osteosynthesis surgery, as well as the corresponding below described system, is of simple design and intuitive use.

Such a guide, as well as system, offers the advantage of requiring only small incisions, and/or tailoring bone sections and correction to the severity of the pathology and the anatomy of the patient, and/or guiding the insertion of implants in a reproducible manner while limiting the number of X-ray images.

Such a guide, as well as system, for example for Lapidus osteosynthesis surgery, allows the preparation of articular surfaces and/or the reduction of bone deformation, and/or the insertion into bone parts of pins guiding the definitive implants.

The guide is for example configured to allow the displacement of one of the two bone fragments relative to the other of the two bone fragments, for example of the second bone fragment, so as to allow at least two degrees of freedom, for example at least one being one in translation, for example at least one in translation and one in rotation, for example at least three or four degrees of freedom, for example two in translation and two in rotation.

The guide, including the correction unit 5, the rotation unit 6, and the aiming unit 7, for example allows for triplanar correction of one of the two bone fragments relative to the other of the two bone fragments, for example of the second bone fragment, for example by allowing at least three degrees of freedom, for example two in translation and one in rotation, for example by allowing four degrees of freedom, for example two in translation and two in rotation.

The two bone fragments are for example foot bones bone fragments. The first bone fragment can be a bone fragment of a first bone, and the second bone fragment can be a bone fragment of a second bone. The first bone and the second bone can present and form a joint inbetween. The first bone fragment can be a bone fragment of an end of the first bone that is proximal to the joint. The second bone fragment can be a bone fragment of an end of the second bone that is proximal to the joint. The first bone and the second bone can be foot bones. The first bone is for example a metatarsal bone, for example the first metatarsal bone. The second bone fragment is for example a cuneiform bone, for example the first cuneiform bone. The joint is for example the cuneo-metatarsal joint, for example the first cuneo-metatarsal joint.

### System

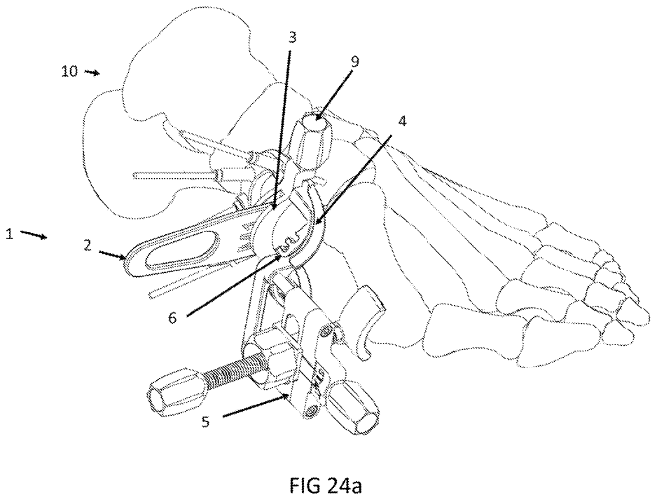

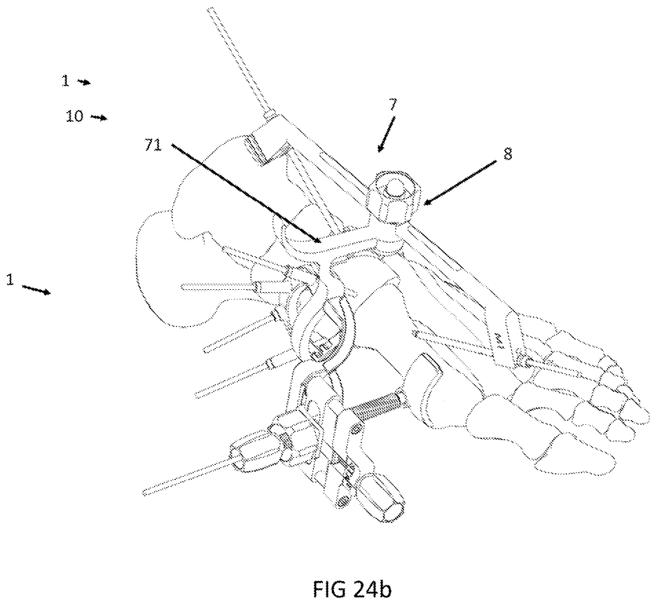

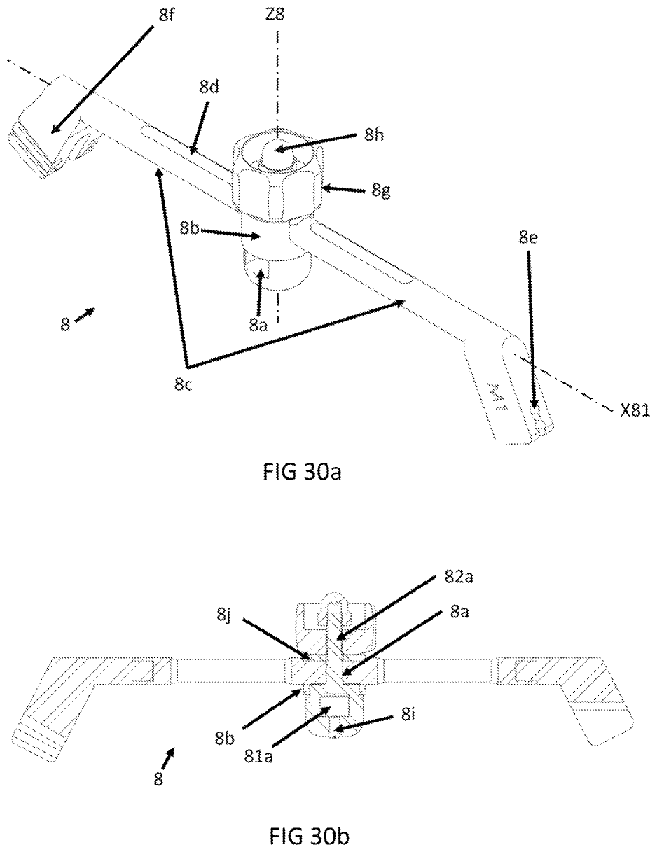

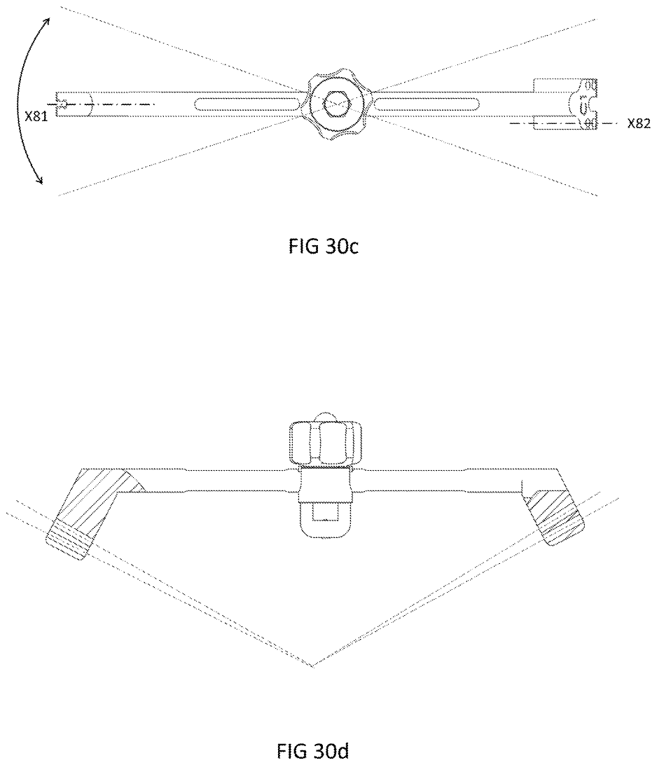

With reference to FIG. 1a to 43, a guide system 1 for example an orthopedic surgical guide system 1 is described. The system 1 is for example an orthopedic surgical guide system for a surgical operation to fuse two bone fragments. The system 1 includes the guide 10.

The system 1 can include a feeler 2 to be inserted between the two bone fragments, the feeler being configured to cooperate with the guide.

The system can include a handle, for example configured to cooperate with a groove of the feeler 2. Alternatively, the feeler 2 can include the handle.

The system can include a plurality of frame attaching pins, for example three frame attaching pins, for example four frame attaching pins, for attaching, for example removably, the frame 4 to the first bone fragment.

The system can include a plurality of lateral junction pins, for example two lateral junction pins. Alternatively, the system can have no such lateral junction pin(s).

The system can include one or a plurality of rotation unit attaching pins, for example one rotation unit attaching pin, for example two rotation unit attaching pins, for example three rotation unit attaching pins, for attaching, for example removably, the rotation unit 6 to the first bone fragment. The rotation unit attaching pin(s) can form rotation unit retaining pin(s).

The system 1 can include a first guiding pin 7i and a second guiding pin 7j.

The system 1 can include a locking pin.

The system 1 can include a first orthopedic implant and/or a second orthopedical implant. The first guiding pin can be configured, when positioned, to guide a first orthopedic implant. The second guiding pin can be configured, when positioned, to guide a second orthopedic implant. The first orthopedic implant can be a screw. the second orthopedical implant can be a screw.

The guide 10 and system 1 can be such that the frame 4, via the cutting window 3 can be centered, for example centered medially, with respect to the joint, for example to the cuneo-metatarsal joint, by insertion of a cutting opening 3b as described hereafter, through the feeler 2 previously been positioned at a center of the joint. The positioning of the feeler 2 at the center of the joint allows this same centering to be transmitted to the frame 4 by means of an opening, for example a feeler guiding opening, for example formed by at least a part of the cutting opening 3b for example the whole cutting opening 3b, and/or a rib guiding opening 3a as described hereafter, of the cutting window 3. Thus, the correct positioning of the frame 4 in relation to the joint, for example the cuneo-metatarsal joint, can for example ensure that the cuts made are always referenced to the joint plane during the bone cutting stage. Such centering means can allow the guide 10 to be removably attached to the second bone fragment, for example the cuneiform bone, thus avoiding incisions between the second and third metatarsals as in the prior art. A first cut is thus made and then a reduction actuator 5a as described hereafter, for example a medio-lateral reduction actuator 5a, is manipulated, which pushes the first bone fragment, for example the metatarsal, laterally. Once the medial-lateral position of the first bone fragment, for example the metatarsal, is adequate, the cut is completed by passing a cutting tool, for example a percutaneous burr as described hereafter, through the same window 3 again.

The system can include a cutting tool, for example at least one or two cutting tool(s), for example for cutting at least one of the two bone fragments. The cutting tool can be a percutaneous burr or a percutaneous drill or a sawblade. The system can include a cutting tool, for cutting the two bone fragments or the system can include a first cutting tool for cutting the first bone fragment and a second cutting tool for cutting the second bone fragment.

### Window

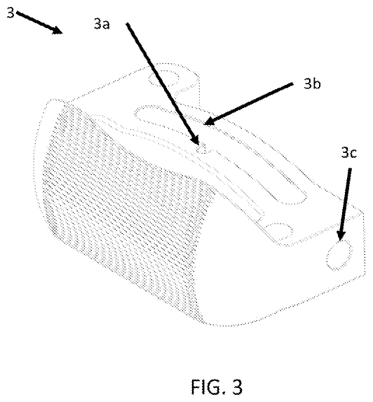

With reference to FIGS. 3, 26, 31, the window 3 is described.

The window 3 can be attached to the frame 4. The window 3 can for example be removably, attached to the frame 4. When removable, the window can be removed, for example to avoid obstructing the visualization in case a medical imaging procedure is performed, for instance by radiology. Such imaging can be performed for example if the operator wants to check that one or several cut(s) as described hereafter has/have been correctly performed and/or to clean an incision bone site located beneath, for instance after the incision has been performed. Alternatively, the window 3 can be attached to the frame 4 in a non-removable way and/or the frame 4 can be attached to the window 3 in a non-removable manner. The window 3 can be integrally formed with the frame 4.

The window 3 can be radiolucent.

The window 3 is for example configured for allowing the passing through and/or guiding and/or insertion, for example therethrough, of at least a part of a cutting tool for cutting one of the two bone fragments. The window can thus have a cutting opening 3b, for example a longitudinal cutting opening 3b, for example a slot 3b, for passage of a cutting tool for cutting one of the two bone fragments. The window 3 can be a percutaneous burr or drill or sawblade cutting window 3.

The window 3 can be configured to allow the insertion of at least a part of a feeler by relative displacement of the window 3 with respect to the feeler 2 so that the feeler guides the frame 4. The window 3 can have a feeler guiding opening for inserting, for example therethrough, of at least a part of a feeler 2 through the guiding opening by relative displacement of the window with respect to the feeler so that the feeler 2 guides the frame 4.

For example, with reference to FIG. 3, the window 3 can have the cutting opening 3b, for example in the form of a slot, and rib guiding opening 3a connected to the cutting opening 3b, for example extending from a central part of the cutting opening 3b, so that the feeler guiding opening is formed by the central part of the cutting opening 3b and the rib guiding opening 3a.

The cutting opening 3b, the rib guiding opening 3a, and/or the feeler guiding opening can extend from an inner face of the window 3 configured to face the first and/or second bone fragments when the window 3 is attached to the frame 4 and the frame 4 is attached to at least the first of the two bone fragments, and/or extend from an outer face of the window 3 configured facing away from the first and/or second bone fragments when the window 3 is attached to the frame 4 and the frame 4 is attached to at least the first of the two bone fragments. The cutting opening 3b, the rib guiding opening 3a, and/or the feeler guiding opening can be through holes extending from the outer face to the inner face of the window 3.

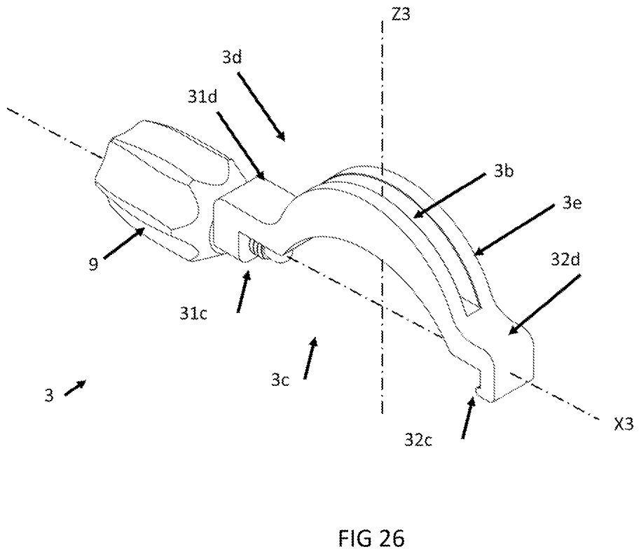

For example, with reference to FIG. 26, the window 3 can have the cutting opening 3b shaped with respect to the shape of the feeler 2, in particular to the width and/or thickness of the feeler 2, so as to form the feeler guiding opening, for example in the form of a slot, for example of a slot only.

The cutting opening 3b, and/or the feeler guiding opening can extend from the inner face of the window 3 configured to face the first and/or second bone fragments when the window 3 is attached to the frame 4 and the frame 4 is attached to at least the first of the two bone fragments, and/or extend from the outer face of the window 3 configured facing away from the first and/or second bone fragments when the window 3 is attached to the frame 4

The window 3 can also include a means of attaching and/or fastening 3c of the window 3 to the frame 4, for example in a removable manner.

With reference to FIG. 3, the means of attaching/fastening 3c can for example present a hole extending from a side face of the window 3. The side face can for example extend between the outer face and the inner face, for example connect the outer face and the inner face for example by means of a pin extending at least partially through the hole.

By inner, it is for example meant facing the first and/or second bone fragments when in use, for example when the guide is attached to the bone fragment. By outer, it is for example meant facing away from the first and/or second bone fragments when in use, for example when the guide is attached to the bone fragment.

By down, it is for example meant relatively inner. By up, it is for example meant relatively outer.

The window 3 can for example be arranged so as to be movable with respect to the frame 4. The window 3 can for example be arranged pivotably with respect to the frame 4, for example with a hinge, for example about an axis of rotation connecting side faces of the window 3, for example passing through the hole of the means of attaching/fastening 3c. That way, the window 3 can be moved for instance so as not to hinder the positioning of the feeler 2 in an optimal manner. The window 3 can for example be lockable in position, for example through an actuator 4i as described hereafter. When the window is locked, the bone cut made by the cutting tool is performed according to the position defined by the feeler. Alternatively, the window 3 can be fixedly secured to the frame 4 so as to avoid any relative movement thereof. That way the guide can be simpler to manufacture and its use requires less operations.

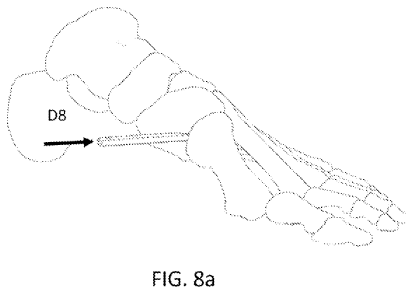

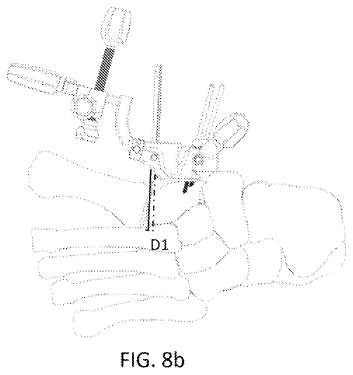

FIG. 8b shows the reference (solid line) created by the feeler 2, within the joint, for example the cuneo-metatarsal joint. The distance between the straight lines, that is to say the solid line the dot-dashed line D1 represents the thickness of the 1st cut with the cutting tool, for example the burr. It can be seen that it is offset on the cuneiform to remove all the cartilage present on it.

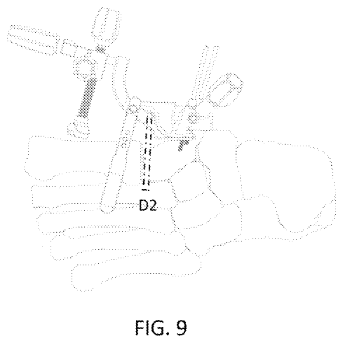

FIG. 9 shows the intermetatarsal angle correction performed as well as the thickness of the second cut D2 that removes the metatarsal cartilage. Thanks to the reduction angle/displacement, the desired bone material on the metatarsal is also removed.

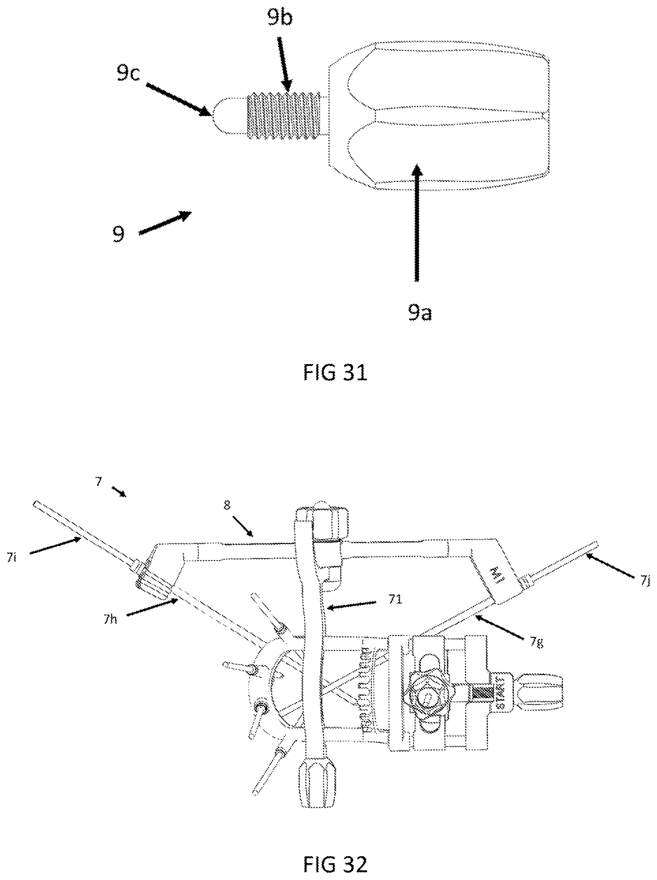

With reference to FIGS. 26, 31, the window 3 can include a window body 3d, the window 3d having the cutting hole 3b.

The means of attaching/fastening 3c can for example present on a first side an attaching/fastening hole 31c, for example a threaded hole, for example extending from a side face of the window 3. The side face can for example extend between the outer face and the inner face, for example connect the outer face and the inner face. The window body 3d can have the hole 31c. The hole 31c is for example circular.

The means of attaching/fastening 3c can further include an attaching/fastening element 9 configured to extend through the hole 31c, the attaching/fastening element 9 being for example a threaded element. This attaching/fastening element 9 can maintain the window 3 in the holes 42b, described hereafter, on the lateral side of the frame 4. The attaching/fastening element 9 can include a threaded shank, including a threaded part 9b, for example threaded portion. The thread of the hole 31c and the thread of the threaded element 9, for example of the threaded part 9b, can correspond.

The attaching/fastening element 9 can include a first end 9a and a second end 9c. The threaded part 9b can be located between the first end 9a and the second end 9c. The first end 9a can be configured to be moved by the practitioner's hand. To this end the first end 9a can be or include a wheel configured to be gripped by the practitioner's hand so as to rotate the attaching/fastening element 9 with respect to the hole 31c, for example so as to translate the second end 9c with respect to the hole 31c, for example with respect to the hook 32c as described hereafter, for example by screwing/unscrewing, for example thanks to the cooperation of the thread of the hole 31c and the thread of the threaded element 9. Alternatively, the means of attaching/fastening 3c could allow for relative translation of the second end 9c with respect to the hole 9c, for example with respect to the hook 32c as described hereafter without the need of a threaded part 9b, by pushing/pulling.

The second end 9c can be shaped so as to interact with the locking hole 42b of the frame as described hereafter, for example so as to allow locking of the window 3 on the frame 4. To this extent, the second end 9c can be shaped so as to allow locking with the locking hole 42b, for example by force fitting.

The means of attaching/fastening 3c can present on a second side, for example a hook 32c extending from a side face of the window 3. The hook 32c can for example maintain the window 3 in grooves 41b as described hereafter, which can be located on a lateral side of the frame 4. The second side is for example a side face of the window 3 facing away from the first side. The hook 32c can form a curve which concavity is turned towards the hole 31c.

Alternatively, the window 3 can be secured in a non-removable way, to the frame 4 to avoid any relative movement thereof. For example, the window 3 and the frame 4 can be integrally formed.

The window 3, for example the window body 3d, can include a curved part 3e having a curvature. The curvature may have a concavity turned towards an inner side when in use, for example facing the first and/or second bone fragments when in use. The cutting hole 3b can be located on the curved part 3e, for example so as to present a curved shape, for example a curved slot shape. The curvature of the curved part 3e is for example configured to allow positioning of the curved part 3e, in particular of the cutting hole 3b, with respect to the patient skin. Alternatively or in combination, The curvature of the curved part 3e is for example configured so as to form a stopper in case of a deep cutting and/or in case of drilling. Alternatively or in combination, the curvature allows for distancing the central part of the cutting hole 3b from the skin when in use. When the window 3 is locked, for example on the frame 4, the bone cut made by the cutting tool is performed according to the position defined by the feeler 2.

The curved part 3e is for example located between a first part of the window body 3d having the hole 31c and a second part of the window body 3d including the hook 32c, for example so that the first part and the curved part 3e form a first shoulder 31d and/or the second part and the curved part 3e form a second shoulder 32d.

### Frame

The frame 4 can be configured to be temporarily attached to at least one of the two bone fragments, for example the first of the two bone fragments. The frame 4 can be configured to be removably attached to a single of the bone fragments to be fused, for example the first bone fragment.

The frame 4 can be configured to rest, for example medially, on the skin extending over the first bone, for example the cuneiform, for example when the frame 4 is attached to the first bone. For example an inner side 4a, or bottom side, of the frame can be configured to rest medially on the skin extending over the first bone, for example the cuneiform, for example when the frame 4 is attached to the first bone. The inner side 4a can present a plurality of holes 4d, for example three holes, for example for attaching, for example fixing, the frame 4, for example the guide 10, to the first bones, for example the cuneiform, for example by means of the frame attaching pins. The frame attaching pins can be threaded. The attaching pins can have stops.

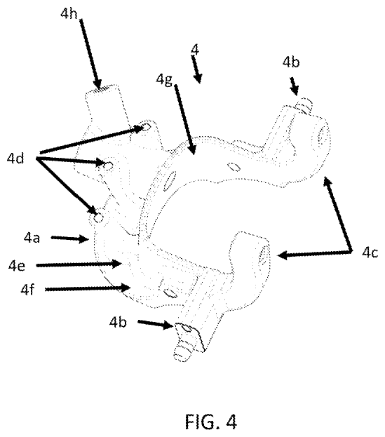

With reference to FIG. 4, the frame 4 can be of an arcuate shape, for example with two arms extending towards a front from a rear part extending in a central part of the arcuate shape inbetween the two arms. The holes 4d can extend from the inner side 4a to an outer side 4e of the frame, for example at a contact part 4f, also called outer part 4f, at the rear of the frame 4. The contact part 4f, or outer part 4f, can follow the arcuate shape of the frame 4. A wall 4g can extend from the contact part 4f, or outer part 4f, for example at a front of the contact part 4f, or outer part 4f. The wall 4g can follow the arcuate shape of the frame 4, defining the two arms of the frame 4.

The frame 4 can include one or several lateral junctions 4b, for example two lateral junctions 4b, each allowing to attach the rotation unit 6 and/or the aiming unit 7 to the frame 4 removably, for example removably both for the rotation unit 6 and/or the aiming unit 7, and for the frame 4. Each lateral junction 4b is for example extending from a different arm of the frame 4, for example extending away from the space defined inbetween the arms. Each lateral junction 4b can for example allow the insertion of one of the lateral junction pins to hold the first bone, for example the metatarsal bone, during the cut of the corresponding bone fragment, and/or also to perform a supination movement to restore the alignment of the first bone fragment, for example the alignment of the metatarsal bone to the sesamoids. Each lateral junction 4b can be configured to be attached to the rotation unit 6, for example alternately, so that the rotation unit 6 can be attached at one side or another of the frame 4. Each lateral junction 4b can be configured to be attached to the aiming unit 7, for example alternately, so that the aiming unit 7 can be attached at one side or another of the frame 4. This allows the guide to be used on symmetrical body parts, for example on the two feet, for example on the right and left first cuneo-metatarsal joints.

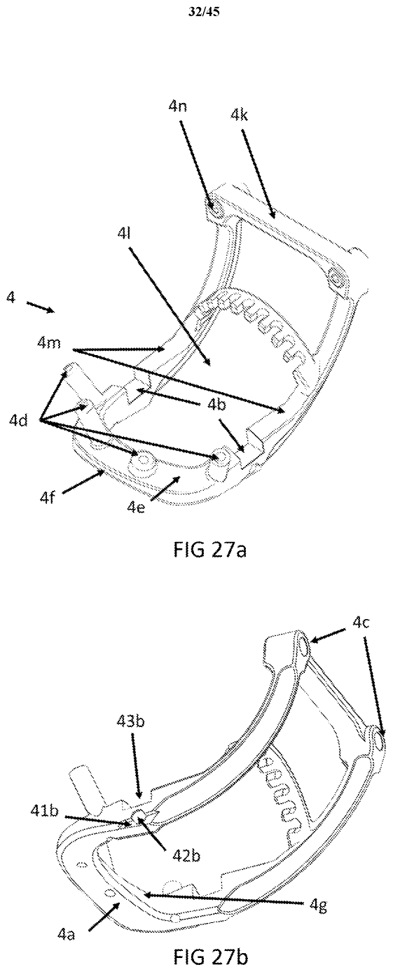

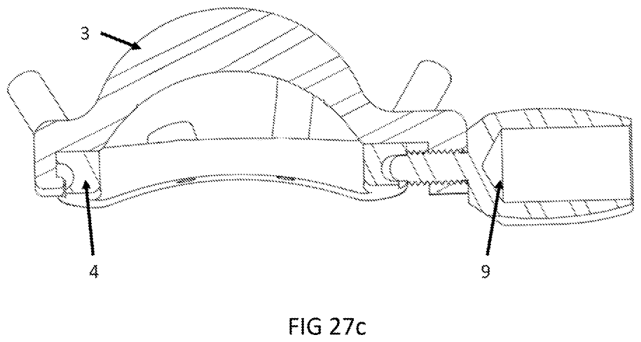

With reference to FIG. 27a-c, the frame 4 can be of an arcuate shape, with two arms 4m extending towards a front from a rear part extending in a central part of the arcuate shape inbetween the two arms 4m. The holes 4d can extend from the inner side 4a to an outer side 4e of the frame, for example at the contact part or outer part 4f at the rear of the frame 4. The outer part 4f can follow the arcuate shape of the frame 4.

At least some of the holes 4d, for example four holes, can be formed by tubular parts extending from the surface of the outer side 4e. One of holes 4d can have a shorter tubular part than the other holes, or no tubular part, so as to allow more anchorage in bone with attaching pins of the same length, for example with same attaching pins.

The frame 4 can be deprived of any wall extending from the outer part 4f toward the upper side, and for example following the arcuate shape of the frame 4. On the contrary, an inner face the arcuate shape of the frame 4 extends from the outer part 4f towards the inner side 4a. The frame 4 can have a “U” shape, the shape being delimited by the inner face/wall 4g so as to allow a good visualization of the incision. The frame 4 can thus by configured so that there is no separation between the outer part 4f and an opening defined by the outer part 4f and the two arms 4m of the frame 4 and allowing access to the incision.

The frame 4 can include one or several lateral junctions 4b, for example two lateral junctions 4b, a first lateral junction and a second lateral junction, the lateral junction(s) 4b allowing to attach the window 3 and/or the aiming unit to the frame 4 removably, for example removably both for the cutting window and/or the aiming unit 7 as described hereafter, and for the frame 4. The lateral junction(s) 4b are for example located on the arm(s) 4m of the frame 4, for example at the connection parts with the outer part 4f. For example the first lateral junction 4b is located on a first arm of the arms 4m of the frame 4, and the second lateral junction 4b is located on a second arm of the arms 4m of the frame 4.

One or more, for example each, of the lateral junctions 4b can have or be formed by a recess 41b, and/or a hole 42b, and/or a groove 43b. The recess 41b is for example configured to receive the hook 32c of the window 3 and/or the hook 71b of the support subunit as described hereafter. The recess 41b is for example located on a wall of the corresponding arm 4m, the wall facing away from the opening 41 defined by the outer part 4f and the two arms 4m of the frame 4. The hole 42b is for example configured to receive the locking threaded wheel 9, the hole 42b opening at least away from the opening 41 defined by the outer part 4f and the two arms 4m of the frame 4. The hole 42b is for example at least partially overlapping the recess 41b, for example at least partially extending within the recess 41b, of the same lateral junction 4b and/or at least partially extending up from the recess 41b of the same lateral junction 4b, relatively at least partially outer than the recess 41b of the same lateral junction 4b.

The groove 43b is for example configured to receive the first shoulder 31d and/or the second shoulder 32d. The groove 43b is for example located on the outer side 4e of the frame 4, for example of the corresponding arm 4m of the frame. The groove 43b is for example extending in a direction transversal to the respective arm 4m. The groove 43b and the hole 42b of a same lateral junction 4b are for example located one above the other in the inner/outer direction.

The frame 4 is for example configured so that the window 3 and/or aiming unit 7 can be attached thereto in at least one position. For example, one of the lateral junctions 4b has such a hole 42b, and the other of the lateral junctions 4b, for example located on the opposite arm of the arms 4m, has such a recess 41b. For example, one of the lateral junctions 4b has such a groove 43b and such a hole 42b, and another of the lateral junctions 4b, for example located on the opposite arm of the arms 4m, has such a groove 43b and such a recess 41b.

Such design allows for an effective locking of the window 3 position before cutting and/or aiming unit 7.

The frame 4 is for example configured so that the window 3 and/or aiming unit 7 can be attached thereto in both positions. For example, each of the lateral junctions 4b has such a hole 42b and such a recess 41b. For example, each of the lateral junctions 4b has such a groove 43b, such a hole 42b, and such a recess 41b. The window 3 can be attached at one side or another of the frame 4, and/or the aiming unit 7 can be attached at one side or another of the frame 4. This allows the guide to be used on symmetrical body parts, for example, on the two feet, for example on the right and left first cuneo-metatarsal joints.

At the end of each of the arms 4m, opposite the contact surface 4f, for example also known as contact part 4f, a protrusion 4c extends, for example towards the outer side. Each lateral junction 4b is for example located between the contact surface 4f and the protrusion 4c of the corresponding arm 4m of the frame 4. The protrusions 4c are configured to allow the correction unit 5 to be attached, for example removably, to the frame 4, for example by accommodating a corresponding part of the correction unit 5.

With reference to FIG. 27a-c, protrusions are for example connected by a bridge 4k. The bridge 4k allows for enhanced rigidity. Protrusion(s) 4c can include an opening for connection to the correction unit 5, in particular to the respective body frame protrusion(s) 5m as described hereafter.

With reference to FIG. 4, the frame 4 can further include a window locking actuator 4i, for example for locking the cutting window. The window locking actuator is for example located at a back of the frame 4, for example on a contact part 4f, for example on a protrusion of the contact part 4f, for example connected to the contact part 4f through a dedicated opening 4h.

### Feeler

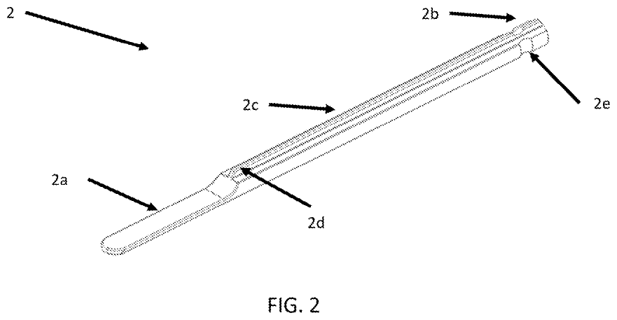

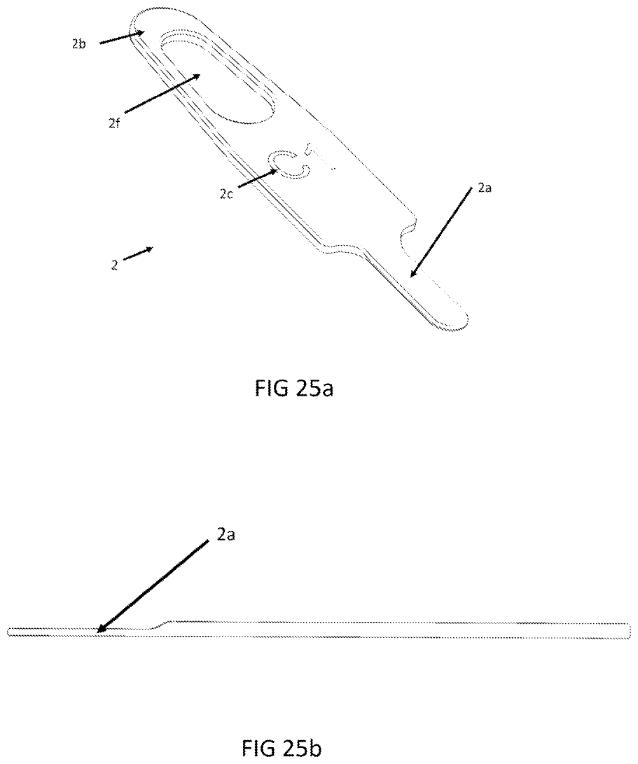

With reference to FIGS. 2, 25a, 25b, the feeler 2 is described. The feeler 2 is for example configured to be inserted between the two bone fragments. The feeler 2 is configured to cooperate with the guide 10. The feeler 2 is for example a joint feeler. The feeler 2 can form a probe.

The feeler 2 can include a first end 2a and a second end 2b. The feeler 2 can extend from the first end 2a to the second end 2b. For example, the feeler 2 can include a central part 2c extending inbetween the first end 2a and the second end 2b, for example connecting the first end 2a and the second end 2b, for example joining the first end 2a and the second end 2b.

The first end 2a can be tapered, for example so as to allow insertion of the first end 2a into the joint.

The central part 2c and/or the second end 2b is/are adapted to guide the window 3, for example so as to guide the frame 4, for example so as to guide the guide 10.

With reference to FIG. 2, the feeler 2 can include a rib 2d, for example extending on the second end 2b and/or on the central part 2c. For example to a distal extremity of the second end 2b up to an extremity of the central part 2c connecting the first end 2a, for example along the part of the feeler that is not tapered. The rib 2d can for example be adapted to cooperate with the guiding opening of the window 3 of the guide 10.

The feeler 2, for example the second end 2b, can have a groove 2e. The groove 2e can extend radially with respect to a longitudinal axis of the feeler 2. The groove can extend from a first groove end at one side of the rib 2d to a second groove end at the other side of the rib 2d.

The groove 2e can be configured to cooperate with the handle. The handle can be a quick-fit handle. The handle allows for manipulating the feeler 2 so as to facilitate insertion of the feeler 2, for example in the joint.

FIG. 8a shows the feeler 2 inserted in the first cuneo-metatarsal joint in the direction D8.

With reference to FIGS. 25a, 25b, the window 3 cutting opening 3b and the feeler 2, in particular the width and/or thickness of the feeler 2, are shaped so that the feeler guides the window 3, as described hereabove.

The feeler 2 can include a hole 2f, provided on the second end 2b and/or on the central part 2c. The hole 2f can be adapted to allow an easier grip by the practitioner's hand, for example a better ergonomic with surgical gloves, and/or facilitate the feeler insertion. The hole 2f can for example have at least a portion widening from the second end 2b towards the first end 2a. The hole 2f can be of an ovoid shape.

The feeler 2 can have two faces, for example defining a flat shape. At least one of the face, for example both faces, for example in the central part 2c, can have a marking so as to allow the feeler 2 insertion in the correct position, for example a laser marking, for example a marking with two fragment bones symbols, for example “C1” for cuneiform, “M1” on the other side for the metatarsal.

The first end 2a of the feeler can have a rounded extremity designed to be inserted. The first end 2a of the feeler can be of relative narrower shape, for example defining narrower faces. The first end 2a of the feeler can be relatively thinner.

The central part 2c of the feeler can be of relative wider shape than the first end 2a, for example defining wider faces than the first end 2a, for example is the widest part of the feeler. The central part of the feeler can be relatively thicker than the first end 2a, for example the thickest part of the feeler. The central part 2c for example is complementary to the shape of the window 3 cutting opening 3b.

The second end 2b of the feeler can be of relative wider shape than the first end 2a, for example defining wider faces than the first end 2a, for example with a width decreasing away from the central part 2c. The second end 2b of the feeler can be relatively thicker than the first end 2a, for example as thick as the central part 2c.

The window 3 and feeler 2 are configured so that the thickness and width of the feeler 2 guides the window 3 when the window is inserted on the feeler 2.

### Correction Unit

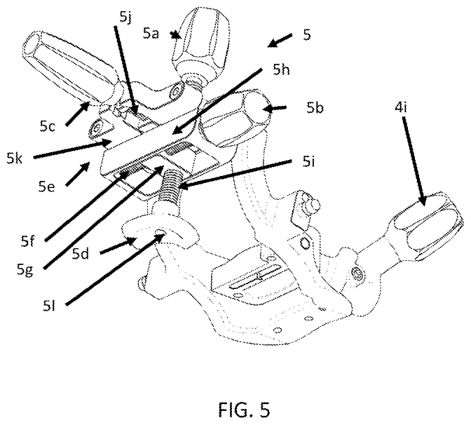

With reference to FIG. 5, 28a-c, the correction unit is described. The correction unit 5 can be a metatarsal position correction unit 5. The correction unit 5 can form a correction block.

The frame 4 and correction unit 5 are for example configured so that the correction unit 5 can be removably attached to the frame 4, for example on the frame 4. Alternatively, the correction unit 5 can be attached to the frame 4 in a non-removable way and/or the frame 4 can be attached to the correction unit 5 in a non-removable manner. The correction unit 5 can be integrally formed with the frame 4.

The correction unit 5 can include a paddle 5d configured to be in contact with one of the two bone fragments, for example the first bone fragment, for example the metatarsal bone fragment.

The correction unit 5 can include a body 5e.

The correction unit 5 can include an elevating/lowering actuator 5b configured to adjust the position of the paddle 5d against the first bone fragment, for example towards the metatarsal in the dorso-plantar direction.

The correction unit 5 can include a reduction actuator 5a for positioning the paddle against the skin before reducing the intermetatarsal angle in the mediolateral direction. The reduction actuator 5a can be connected to the paddle 5d through a rod 5i, for example the reduction actuator 5a and the paddle 5a being located at to opposite ends of the rod 5i. The rod 5i can include a threaded portion cooperating with a corresponding internal thread of the body 5e of the correction unit 5, for example so that rotation of the reduction actuator 5a results in elevating/lowering the paddle 5d. The correction unit 5 can be configured so as to allow reducing an angle of the first bone with a neighboring bone, for example the intermetatarsal angle, through a rotation of the first bone with respect to the neighboring bone, by actuating the reduction actuator 5a so as to move the paddle 5d, for example while a rotation unit attaching pin has been positioned in one of the attaching pin openings 6e, and so as to be attached to the first bone fragment.

The reduction actuator 5a can extend from a first end of rod 5i, and can include a wheel, for actuation by a practioner's hand. The rod 5i can have a second end, for example of reduced diameter so as to be inserted in the paddle 5d.

The paddle 5d can include an arcuate part designed to be in contact with the skin when in use. The paddle 5d includes an outer part designed to receive the second end of the rod 5i. The outer part is located in a central portion of the paddle 5d, the central portion being for example bored, the bore extending through both arcuate part and outer part, the bore having for example a first section of gradually reducing size, for example diameter, from the inner side towards the outer side, followed in the same sense with a second part forming a recess for the second end of the rod 5i.

With reference to FIG. 5, a rod 5f, for example a threaded rod 5f, can be connected to the elevating/lowering actuator 5b, for example extend from the elevating/lowering actuator 5b. The rod 5f can extend through a first movable unit 5g of the body 5e, the first movable unit 5g being configured to move within a first body frame 5h of the body 5e, upon actuation of the elevating/lowering actuator 5b, for example upon rotation of the elevating/lowering actuator 5b. The rod 5i is for example extending through the first movable unit 5g, so that the displacement of the first movable unit 5g within the first body frame 5h by actuation of the elevating/lowering actuator 5b results in the corresponding displacement of the rod 5i and therefore of the paddle 5d. Through connection of the paddle with the first bone fragment, actuation of the elevating/lowering actuator 5b allows elevating/lowering the first bone fragment with respect to the second bone fragment.

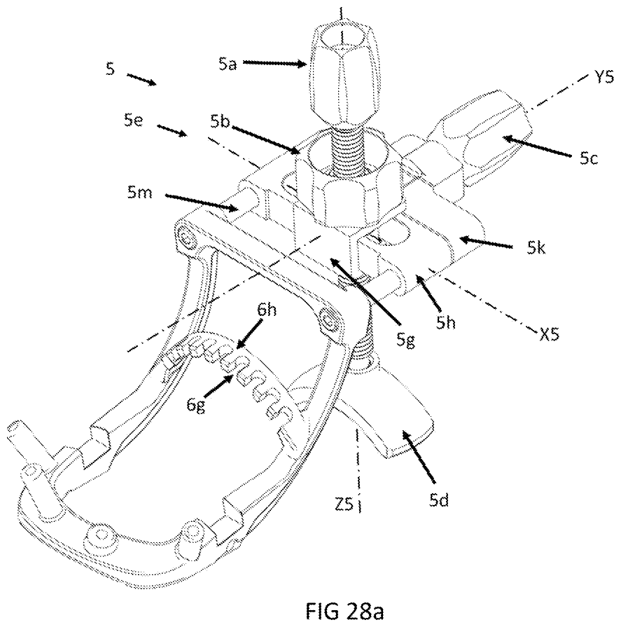

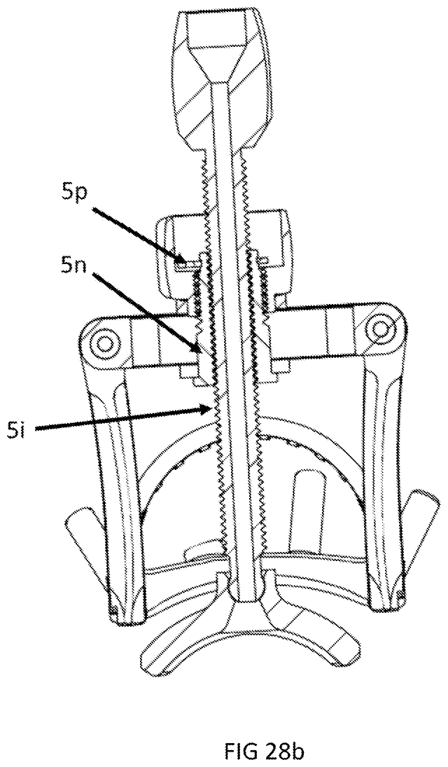

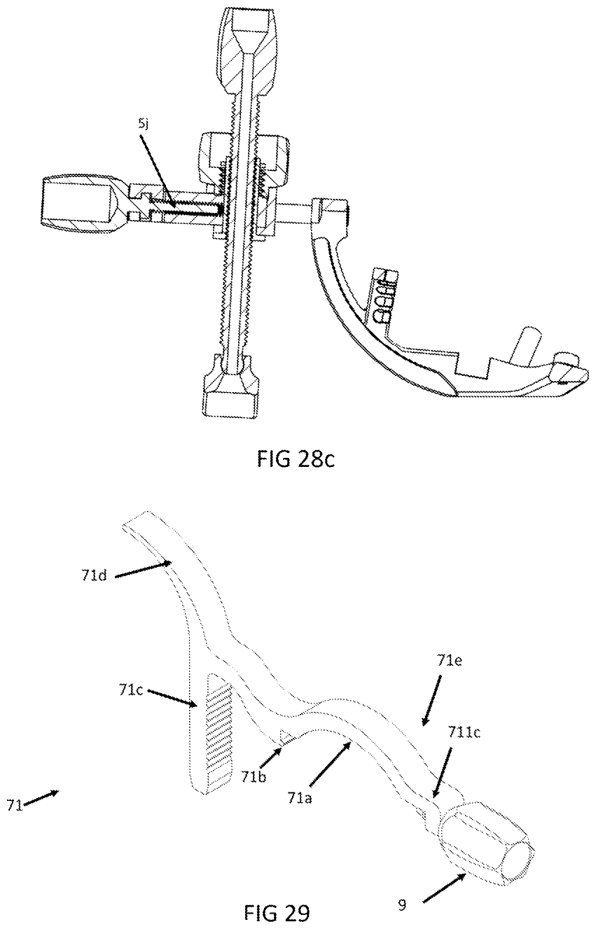

With reference to FIG. 28a-c, the rod 5i, for example the threaded rod 5i, can be connected to the elevating/lowering actuator 5b, for example extend through the elevating/lowering actuator 5b. The elevating/lowering actuator 5b can be of the shape of a wheel with a central opening through which the rod 5i can extend and connect to the elevating/lowering actuator 5b. The rod 5i can extend through a first movable unit 5g of the body 5e, the first movable unit 5g being configured to move within a first body frame 5h of the body 5e, upon actuation of the elevating/lowering actuator 5b, for example upon rotation of the elevating/lowering actuator 5b. The rod 5i is for example extending through the first movable unit 5g, so that the displacement of the first movable unit 5g within the first body frame 5h by actuation of the elevating/lowering actuator 5b results in the corresponding displacement of the rod 5i and therefore of the paddle 5d. Through connection of the paddle with the first bone fragment, actuation of the elevating/lowering actuator 5b allows elevating/lowering the first bone fragment with respect to the second bone fragment. The first movable unit 5g can form a recess. The first movable unit 5g can include or have a “U” shape. The first movable unit 5g can have a side vertical section which outer end is connected to a horizontal outer wall, and which inner end is connected to a horizontal inner wall, so as to form the “U” shape. A section of the body frame 5h, for example sections of two arms of the body frame 5h, can for example extend with the recess formed by the first movable unit 5g, the two arms connecting two ends of the body frame 5h and defining therebetween an opening along which the rod 5i can move. The elevating/lowering actuator 5b is for example located on top of the first movable unit 5g, for example between the first movable unit 5g and the reduction actuator 5a. Rod 5i is therefore actuated by reduction actuator 5a and elevating/lowering actuator 5b.

The correction unit 5 can include connection element 5n, connection element 5n including or forming a bored cylinder, for example shouldered, for example so as to form a bore, the bore being for example threaded, for example so as to form a thread configured to cooperate with the rod 5i. Connection element 5n can have a threaded outer portion, for example so as to cooperate with elevating/lowering actuator 5b.

Connection element 5n, can form a groove, for example a lateral groove, for example on its end located towards the outer side, for example on its end cooperating with the elevating/lowering actuator 5b, for example adapted to receive a ring 5p, for example a snap ring, so as to retain the elevating/lowering actuator 5b.

Elevating/lowering actuator 5b, for example in cooperation with connection element 5n, can form a recess, for example facing away from the inner side, for example the ring 5p.

Connection element 5n is for example attached to body frame 5h, connection element 5n passing for example through body frame 5h.

The reduction actuator 5a can have a shaft or bore passing therethrough. The shaft or bore can pass through the rod 5i and/or the paddle 5d, for example the shaft or bore being an open shaft or bore, connecting the reduction actuator 5a and the paddle 5d, opening on the reduction actuator 5a and on the paddle 5d. As a result, the device formed by the reduction actuator 5a, the rod 5i and the paddle 5d can be cannulated.

The correction unit 5 can include a compression actuator 5c for bringing the first bone fragment, for example the metatarsal, closer to the second bone fragment, for example to the cuneiform, for example in the antero-posterior direction. A rod 5j, for example a threaded rod 5j, can be connected to the compression actuator 5c, for example extend from the compression actuator 5c. The rod 5j can extend through a second movable unit, the second movable unit including or being for example the first movable unit 5g of the body 5e and/or the first body frame 5h or another movable unit of the body 5e. The second movable unit is for example configured to move within a second body frame 5k of the body 5e, upon actuation of the compression actuator 5c, for example upon rotation of the compression actuator 5c. The rod 5j is for example extending through the second movable unit, so that the displacement of the second movable unit within the second body frame 5k by actuation of the compression actuator 5c results in the corresponding displacement of the rod 5i and therefore of the paddle 5d. The second body frame 5k can for extend include two second body frame protrusions 5m for attachment to the frame 4, for example each second body frame protrusion is configured to be attached to the corresponding protrusion 4c of the frame 4, for example to be accommodated within the corresponding protrusion 4c of the frame 4.

One or several of, for example each of, the body frame protrusions can form a shaft, for example a tubular shaft. The shaft can include or be made of a cylinder, the cylinder having for example a bored side, for example bored and threaded housed within the second body frame 5k.

The body frame 5h is for example configured to slide along the body frame protrusions, upon actuation of compression actuator 5c, by the way of bores of the body frame 5h designed to receive at least a section of the body frame protrusions 5m.

The compression actuator 5c can extend from a first end of rod 5j, and can include a wheel, for actuation by a practioner's hand. A shoulder of the second body frame 5k allows for maintaining the rod 5j.

### Rotation Unit

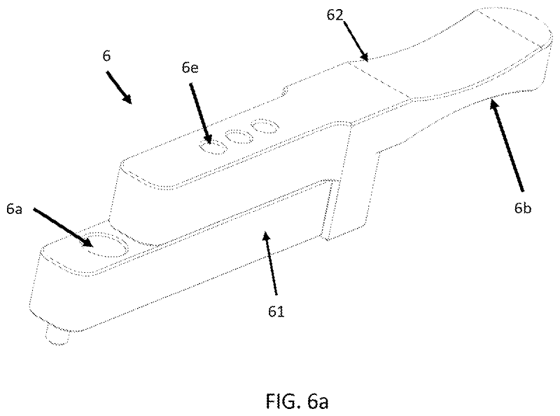

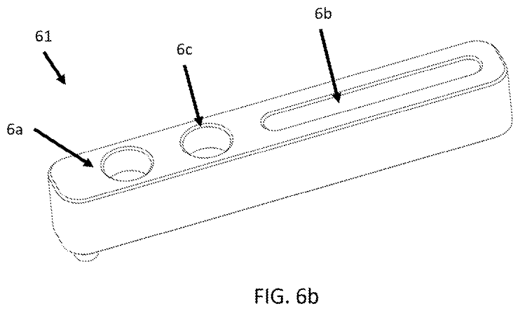

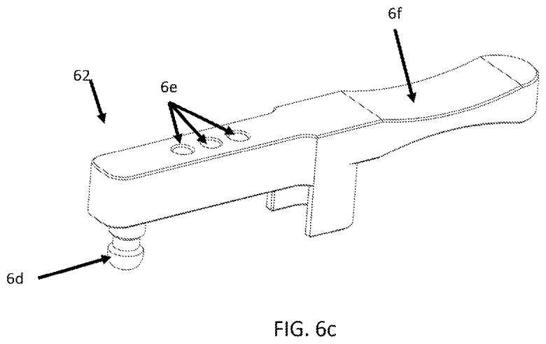

With reference to FIGS. 6a to 6c, 28a to 28c, the rotation unit is described.

With reference to FIGS. 6a to 6c, the frame 4 and the rotation unit 6 are for example configured so that the rotation unit 6 can be removably attached to the frame 4. The rotation unit 6 can form a rotation arm. The rotation unit 6 can form a rotation block.

The rotation unit 6 has at least one attaching pin opening 6e, for example several attaching pin openings 6e, for example three attaching pin openings 6e. Each attaching pin opening 6e can be configured to allow passage of a rotation unit attaching pin so as to allow the rotation unit to be attached, through the rotation unit attaching pin(s), to the first bone fragment, for example to a proximal end of the first bone fragment, for example to a proximal end of the metatarsal.

The rotation unit 6 can be used to adjust the supination of the first bone fragment, for example of the metatarsal.

The rotation unit 6 can include a base 61 and/or a cap 62. The base 61 can be configured to be attached to one of the lateral junction(s) 4b of the frame 4 by means of a connection socket 6a of the base 61.

The base 61 can have a housing 6c, which is for example cylindrical.

The base 61 can have an oblong slot 6b through which the rotation unit fixation pin can be passed to be fixed in the proximal part of the metatarsal. The oblong slot 6b can for example form an opening configured to accommodate one or several of the attaching pins to be used as described above. To this extent, the oblong slot 6b and the attaching pin opening(s) 6e can be configured to be aligned, in particular when the base 61 and the cap 62 are assembled together.

The base 61 can be, for example removably, surmounted by the cap 62. The cap 62 can include a connection socket 6d for assembling the cap 62 and the base 61. To this extent, the connection socket 6d can cooperate with the housing 6c, for example the housing being configured to accommodate the connection socket. The connection socket can form a protrusion extending from a body of the cap 62.

The cap 62 can present the attaching pin opening(s) 6e. An anterior area of the cap can present the attaching pin opening(s) 6e. The attaching pin opening(s) 6e can form holes. The rotation unit 6 can be configured in such a way that a rotation unit attaching pin passing through one of the attaching pin opening(s) 6e will also pass through the oblong slot 6b. A back area of the cap 62 forms a holding/handling area 6f for holding/handling by an operator.

The rotation unit 6 is for example used during the surgical step of obtaining a supination movement of the first bone fragment, for example of the metatarsal. For example, when the rotation unit 6 is attached to the lateral junction 4b of the frame 4 by the connection socket 6a, each of the attaching pin opening(s) 6e allow the passage of a rotation unit attaching pin, thus uniting the position of the first bone fragment, for example of the metatarsal, for the bone cut and also of the center of rotation for the first bone fragment, for example metatarsal, reduction, upon performing a rotation of the first bone fragment upon actuation of the reduction actuator 5a. The presence of several attaching pin openings 6e allows for positioning the pin in the metatarsal bone while the size of the foot can vary.

Further, when the rotation unit 6 is attached to the lateral junction 4b of the frame 4 by the connection socket 6a, the oblong slot 6b allows the passage of the same rotation unit attaching pin, which can be used for the supination of the first bone fragment, for example of the metatarsal.

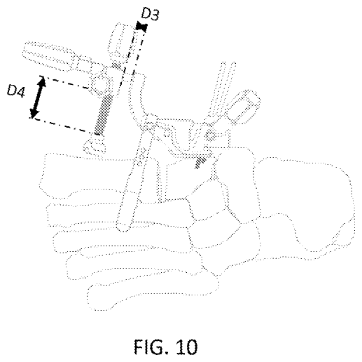

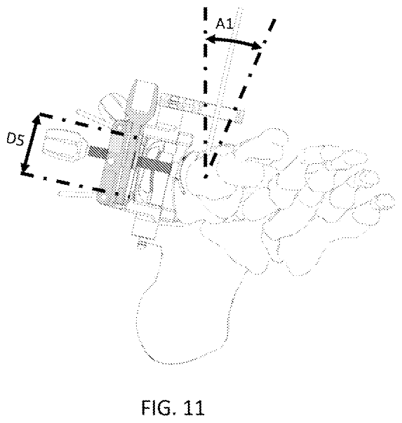

FIGS. 10 and 11 show each possible metatarsal correction and rotation movements distinguished separately. The dimensions D3 and D4 of FIG. 10 show the possible strokes of the compression and reduction movements, while the dimensions D5 and the angle A1 of FIG. 11 represent the elevation/lowering stroke and the possible degree of rotation of the correction unit 5 and the rotation unit 6. A cut-view out is present on the correction unit 5 and on the rotation unit 6.

With reference to FIG. 28a to 28c, the frame 4 and the rotation unit 6 are configured so that the rotation unit 6 can be removably or non-removably attached to the frame 4 for example positioned on the medial side. For example, the rotation unit 6 is to remain attached to the frame 4. For example, the frame 4 and the rotation unit 6 are formed integrally with each other, for example in one single piece.

The rotation unit 6 can form a rotation arc 6h, for example by including an arcuate shape, for example the arcuate shape having a concavity turned towards the inner side. The rotation unit 6 can have one or more teeth and/or one or more grooves, for example so as to form a rotation comb.

The rotation unit 6 has thus at least one attaching pin groove 6g, for example several attaching pin grooves 6g, for example nine attaching pin grooves 6g. The grooves can be configured to allow the attaching pin(s) to pass therethrough, and to allow the metatarsal rotation. The grooves 6g can be configured to retain the attaching pins into the holes.

The grooves 6g can extend through the rotation arc 6h so as to connect the inner and outer sides of the rotation arc. The grooves 6g can define a bottom thereof and lateral walls thereof. The grooves 6g are for example arranged so that their lateral walls extend away from the correction unit 5 when connected to the frame 4, and extend towards outer part 4f and/or inner face/wall 4g. The grooves 6g are for example arranged so that their bottom is oriented towards the correction unit 5 when connected to the frame 4, with respect to their lateral walls.

Each attaching pin groove 6g can be configured to allow passage of a rotation unit attaching pin to allow the pin to be attached, through the rotation unit 6 so as to be attached to the first bone fragment, for example to a proximal end of the first bone fragment, for example to a proximal side of the metatarsal.

The rotation unit 6g can be used to adjust the supination of the first bone fragment, for example of the metatarsal with the aid of the rotation arc 6h geometry of the comb. The rotation unit 6 is used during the surgical step of obtaining a supination movement of the first bone fragment, for example of the metatarsal.

The attaching pin groove(s) 6g allow the passage of a rotation unit attaching pin, thus uniting the position of the first bone fragment, for example of the metatarsal. The presence of several attaching pin grooves 6g allows to choose a supination angle of the first bone fragment, for example of the metatarsal.

The rotation arc 6h can extend from the frame 4, for example from one or the arms of the frame 4. The rotation arc 6h can have two ends, one or each being in contact with a respective arm of the frame 4, for example in a central part of the respective arm, for example between the respective lateral junction 4b and the respective protrusion 4c.

The guide 10 and system 1 can allow for precise centering of the position of the bone cuts made by positioning the feeler 2 in the joint which serves as an anatomical reference. The guide 10 and system 1 can further allow minimally invasive attaching of the guide and only on the second bone, for example the cuneiform, thus avoiding an additional incision, for example between the second and third metatarsals. The guide 10 and system 1 can further allow precise adjustment of the bone correction, for example the metatarsal correction, in all directions thanks to the corrector unit 5, which also integrates joint compression.

### Aiming Unit

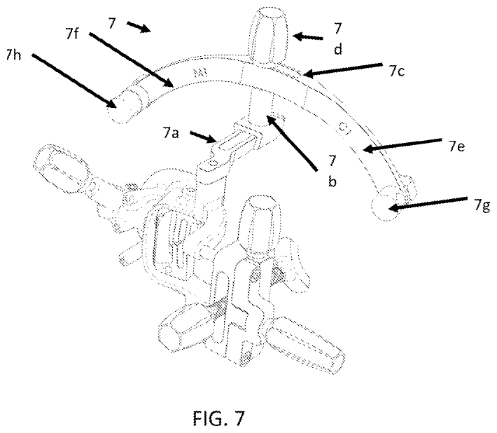

With reference to FIG. 7, 29, 30a-d, 31, 32, the aiming unit 7 is described. The frame 4 and aiming unit 7 can be configured so that the aiming unit 7 can be removably attached to the frame 4.

With reference to FIG. 7, the aiming unit 7 can be configured to be attached to one of the lateral junction(s) 4b of the frame 4 by means of a connection socket of the aiming unit 7. The aiming unit 7 can form an aiming block.

The rotation unit 6 can thus be replaced by the aiming unit 7.

The aiming unit 7 can include at least one slide 7b, for example a pivot slide 7b. For example, the aiming unit 7 can include a sliding arm 7a, the pivot slide 7b being for example inserted on the sliding arm 7a, so that the pivot slide 7b is configured to slide along the sliding arm 7a.

The aiming unit 7 can further include an aiming arch 7c, for example located above the pivot slide 7b, for example so that the pivot slide 7b is located between the frame 4 and the aiming arch 7c. The aiming arch 7c can for example allow the orientation and adjustment of the cross mounting of the implants, for example the arthrodesis screws.

The aiming unit can include a first arm 7e and a second arm 7f, the first arm 7e including a first housing configured for receiving a first barrel 7g, the second arm 7f including a second housing configured for receiving a second barrel 7h. The aiming arch 7c can include the first arm 7e and the second arm 7f, the first arm 7e and the second arm 7f being for instance each of an arcuate shape.

The guide can further include the first barrel 7g and the second barrel 7h, for example such that, when the first housing receives the first barrel 7g, the first barrel 7g is configured to receive the first guiding pin and the second barrel is configured to receive the second guiding pin, so as to position the first guiding pin and the second guiding pin with respect to at least one of the bone fragments. The first guiding pin can be configured, when positioned, so as to guide the first orthopedic implant, and the second guiding pin can be configured, when positioned, so as to guide the second orthopedic implant.

The first barrel 7g can be a drill barrel. The second barrel 7h can be a drill barrel. The first and second housings for drill barrels can be located at both ends of the aiming arch 7c.

The aiming unit 7 can include a locking actuator 7d. The locking actuator can for example extend from the pivot slide 7b, for example so that a connecting body of the pivot slide 7b extends through a slide of the aiming arch 7c. The slide can be on the upper side of the aiming arc 7c. A final selected position of the aiming arch 7c with respect to the locking actuator 7d and/or of the pivot slide 7b with respect to the sliding arm 7a can be locked by means of the actuator 7d, for example before insertion of the first and/or second guiding pin(s) that are to guide the first and/or second orthopedic implant(s).

For example, interaction of the sliding arm 7a with the pivot slide 7b allows for a movement following the shape of the corresponding slide, for example a translation, the extension of the movement depending on the size of the second bone fragment. For example, actuating locking actuator allows a rotation of the aiming arch 7c about the longitudinal axis of the locking actuator 7b, for example the axis connecting the locking actuator 7d and the pivot slide 7b. For example, interaction of the locking actuator 7d and the aiming arc 7c allows a rotation about an axis orthogonal to the axis connecting the locking actuator 7d and the pivot slide 7b.

The guide 10 and system 1 can further allow easy positioning of the implants, for example screws, for example in a cross pattern, with the help of the aiming unit 7.

With reference to FIG. 29, 30a-d, 31, 32, the aiming unit 7 can be configured to be attached to the frame 4, for example to one of the lateral junction(s) 4b of the frame 4, for example by means of an attaching/fastening element 9, for example a connection actuator, and/or a hook 71b. The window 3 can thus be replaced by the aiming support 7.

The aiming unit 7 can include a support subunit 71 and an aiming subunit 8. The support subunit 71 can be configured to be attached to the frame 4, for example to one of the lateral junction(s) 4b of the frame 4, for example by means of the attaching/fastening element 9 and/or the hook 71b.

The attaching/fastening element 9 can be the attaching/fastening element 9 as described hereabove with the same reference, in which case the same attaching/fastening element can be used at two separate occasions. Alternatively, the attaching/fastening element can be another attaching/fastening element with respect to the attaching/fastening element 9 as described hereabove with the same reference, in which case the two attaching/fastening element can be identical or different. In any case the above description of the attaching/fastening element 9 can still apply.

For example, the support subunit 71 can include a first arm, for example an attachment arm, the attachment arm being for example a sliding arm 71c. The arm 71c can include one or several grooves, and/or teeth, for example so as to form an indentation, to block the translation movement, for example in cooperation with a spring plunger 8i, for example a ball spring plunger, of the aiming subunit. The grooves, for example teeth, for example indentation, can be located on a side of the arm 71c turned towards the second arm 71e, for example the curved part 71a, as described hereafter.

The support subunit 71 can have a central part. The central part can be located on a second arm 71e of the support subunit 71, for example a targeting arm 71e, extending from the first arm 71c. The support subunit 71 can have a curved part 71a. The targeting arm 71e, for example the central part, which forms a central part of the targeting arm 71e, can include the curved part 71a. The curvature of the curved part 71a on the central part of the targeting arm 7 is arranged so as to respect the skin curvature of the bone fragment and preserve it, when in use. The curvature of the curved part 71a has a concavity for example oriented towards the inner side when in use, for example facing the first and/or second bone fragments when in use.

The curved part 71a is for example located between the proximal part of the second arm 71e and a distal part of the second arm 71e, for example so that the proximal part and the curved part 71a form a shoulder and/or the distal part and the curved part 71e form a shoulder.

The support subunit 71 can include a third arm 71d, for example a curved arm 71d to facilitate the targeting arm 71e installation by facilitating grasping of the support subunit 71 by the practitioner's hand by means of the curved arm 71d. The curved arm can face away from the second arm 71e, the curvature having for example a concavity oriented towards the inner side. The third arm 71d can have a decreasing dimension from a proximal part thereof to a distal part thereof, for example with respect to the first arm 71c.

Alternatively, the support subunit 71 can be deprived of such third arm 71d. In such case, the practitioner's hand will hold the support subunit 71 through first arm 71c or second arm 71e.

The second arm 71e can include a proximal part and a distal part.

The second arm 71e can for example present on a first side an attaching/fastening hole 711c, for example a threaded hole. The hole 711c is for example circular.

The aiming unit can further include the attaching/fastening element 9 configured to extend through the hole 711c, the attaching/fastening element 9 being for example a threaded element. This attaching/fastening element 9 can maintain the support subunit 71 in the hole(s) 42b, described hereabove, on the lateral side of the frame 4. The attaching/fastening element 9 can include a threaded shank, including a threaded part 9b, for example threaded portion. The thread of the hole 31c and the thread of the threaded element 9, for example of the threaded part 9b, can correspond.

The attaching/fastening element 9 can include a first end 9a and a second end 9c. The threaded part 9b can be located between the first end 9a and the second end 9c. The first end 9a can be configured to be moved by the practitioner's hand. To this end the first end 9a can be or include a wheel configured to be gripped by the practitioner's hand so as to rotate the attaching/fastening element 9 with respect to the hole 711c, for example so as to translate the second end 9c with respect to the hole 711c, for example with respect to the hook 71b, for example by screwing/unscrewing, for example thanks to the cooperation of the thread of the hole 711c and the thread of the threaded element 9. Alternatively, the means of attaching/fastening 3c could allow for relative translation of the second end 9c with respect to the hole 9c, for example with respect to the hook 71b as described hereafter without the need of a threaded part 9b, by pushing/pulling.

The second end 9c can be shaped so as to interact with the locking hole 42b of the frame as described hereabove, for example so as to allow locking of the aiming unit 7 on the frame 4. To this extent, the second end 9c can be shaped so as to allow locking with the locking hole 42b, for example by force fitting.

The proximal part of the arm 71e can present the hook 71b extending hole 711c. The hook 711b can for example maintain the aiming unit 7 in grooves 41b as described hereabove, which can be located on a lateral side of the frame 4. The hook 711b can form a curve which concavity is turned towards the hole 711c.

The aiming subunit 8 can be inserted on the support subunit 71, for example with respect to a connecting element 8a of the aiming subunit 8. The connecting element 8a is for example configured to be inserted on the sliding arm 71c, for example by means of an opening, for example a sliding slot 81a, of the connecting element 8a of complementary shape of the sliding arm 71c, so that the connecting element 8a is configured to slide along the sliding arm 71c. The spring plunger 8i of the aiming subunit 8 can be inserted into a dedicated opening, for example a bore opening on the inner side, of the connecting element 8a, for example to allow maintaining in position the connecting element 8a on the sliding arm 71c.

The support subunit 71 and the aiming subunit 8 can be removably attached to each other. Alternatively, the support subunit 7, in particular the first arm 71c, and the connecting element 8a can be formed integrally, for example made of a single piece.

The aiming unit 8 can further include one or several, for example two, aiming arm(s) 8c, located on the connecting element 8a, for example on a central pivot 82a of the connecting element 8a, the central pivot 82a extending for instance in the direction defined from the inner side to the outer side when in use. The central pivot 82a can extend from a base of the connecting element 8a forming the sliding slot 81a. The aiming arm(s) 8c can allow the orientation and adjustment of the cross mounting of the implants, for example the arthrodesis screws. The aiming arms 8c are for instance arranged on opposite lateral sides of the central pivot 82a. The central pivot 82a can include a threaded rod, for example shouldered at its basis proximal to the targeting guide opening for the first arm 71c.

The aiming subunit 8 can include a first housing 8e and a second housing 8f, for example each arranged on a distal end of a respective aiming arm 8c with respect to the connecting element 8a, for example fixed, for example by welding, so as to ensure their position.

The first housing 8e can include several holes, for example at least two openings holes, for example exactly two holes, corresponding to different angulations, configured for receiving the first barrel. The holes, for example the two holes, are for example connected through a groove opening on a surface of the first housing for example oriented towards the inner side. The groove facilitates dismantling of the guide when setting the pins, for example the groove being large enough to let the pins through.

The second housing 8f can include several holes, for example at least four holes, for example exactly four holes, configured for receiving the second barrel. The four holes are for example arranged so as to vary angulation and gap inbetween pins and/or to select the lateral and/or position of the first pin. The holes are for example arranged by sets of two on lateral sides of the housing. A groove is opening on a surface of the second housing for example oriented towards the inner side. The groove is for example arranged in between the holes, without passing through any of these holes. The groove facilitates dismantling of the guide when setting the pins, for example the groove being large enough to let the pins through.

The guide can further include the first barrel 7g and/or the second barrel 7h, for example such that, when the first housing 8e receives the first barrel 7g, the first barrel 7g is configured to receive the first guiding pin 7i and the second barrel 7h is configured to receive the second guiding pin 7j, so as to position the first guiding pin and the second guiding pin with respect to at least one of the bone fragments. The first guiding pin can be configured, when positioned, so as to guide the first orthopedic implant, and the second guiding pin can be configured, when positioned, so as to guide the second orthopedic implant.

The first barrel 7g can be a drill barrel. The second barrel 7h can be a drill barrel. The first and second housings for drill barrels can be located at both ends of the aiming arch 7c.

The plurality of the holes allows a large orientation choice of the wires/guiding pins, then screws for the practitioner. The first housing 8e and the second housing 8f, can for instance be tilted, for example towards the inner side when in use, for example arcuate, with respect to a respective proximal end of the respective aiming arm.

The first guiding pin can be configured, when positioned through a hole of the aiming unit 8, to guide the first orthopedic implant, and the second guiding pin can be configured, when positioned through a hole of the aiming unit 8, so as to guide the second orthopedic implant.

The aiming arm(s) 8c are for example mounted rotating with respect to the central pivot 82a of the connecting element 8a. The aiming subunit can include a locking mechanism to lock the rotation of at least one or all of the aiming arm(s) 8c with respect to the central pivot 82a and/or translation with respect to the first arm 7c.

The locking mechanism can include a locking actuator 8g, for example a wheel with a threaded bore mounted on the thread of the central pivot 82a, for example so that the proximal part of the aiming arm(s) 8c is positioned, along the central pivot 82a, between the basis of the connecting element 8a and the locking actuator 8g, so as to allow locking by the actuator, for example by rotating the locking actuator 8g. The locking mechanism can include a washer 8j located between the aiming arm(s) and the locking actuator 9g, so as to avoid rotation of the aiming arm(s) upon locking by rotating the locking actuator 9g. The locking mechanism can further include a ring 8b arranged so that, upon rotation of the locking actuator 8g, the ring 8b can be lowered so as to enter into contact with the first arm 7c so as to block its relative sliding and/or translation.

The aiming arm(s) 8c are for example cylinder-shaped.

The aiming arm(s) 8c can, for example each, include a groove 8d, for example extending along a longitudinal direction of the respective arm, the groove 8d forming for example a slot passing through the relative aiming arm so as to open on both sides thereof. The groove(s) 8d allow to visualize the positioning of the pin under X-ray and/or to make the corresponding part lighter.

The aiming subunit 8 can include bolt 8h configured to be mounted on the central pivot 82a, so as to avoid dismantling of the aiming subunit 8, while allowing a constant play to facilitate the setting of the aiming arm(s) 8c.

### Percutaneous Burr

The system can include a percutaneous burr. Such a burr, used in combination with the guide, makes the preparation phase much less invasive than with current techniques based on the oscillating saw.

### Method

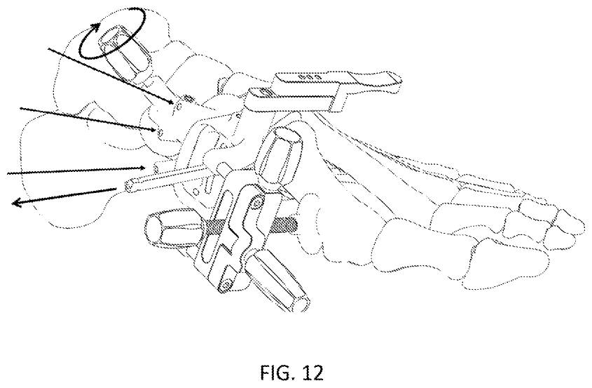

With reference to FIGS. 12 to 23, 33 to 43 a surgical method is described. The surgical method is implemented by the guide 10 and/or the system 1. The surgical method is for example a surgical method to fuse the two bone fragments.

The method can include a first part, for example a first series of steps, for performing cut(s) on the first and/or second bone fragment(s).

The method, for example the first part or series, can include a first step of locating the joint, for example the cuneo-metatarsal joint, for example in a medial area of the joint.

The first step includes creating an incision, for example a vertical incision. The incision can be of a few millimeters. The incision can be created, for example by medial approach, using a cutting element, for example a scalpel.

The first step can include, for example after the creating, at least partially inserting the feeler 2, for example inserting the front end 2a of the feeler 2, for example well medial, into the incision, for example at a bottom of the joint. Once positioned, the feeler 2 can be left in position to serve as a reference (see for example FIG. 8a).

The method, for example the first part or series, can include, for example after the first step, a second step of positioning the frame 4, for example with the cutting window 3, for example the cutting window 3 being attached to the frame 4, for example supporting the cutting window 3. The second step can include preliminarily ensuring the “initial”position of the frame before positioning it.

The second step can include positioning the frame 4 is placed by guiding it over the feeler 2, for example through the feeler guiding opening and/or the rib guiding opening 3a of the cutting window 3. The cutting window 3 can be guided by the rib 2d until the inner side 4a of the frame 4 touches one of the two bone fragments, for example the second bone fragment, for example a medial side thereof, for example the medial side of the cuneiform.

The method, for example the first part or series, can include, for example after the first step and/or second step, a third step (see FIG. 12).

The third step can include of inserting one or a plurality of the frame attaching pin(s), for example three frame attaching pins, for example three such threaded pins with stops, into the hole(s) 4d provided to hold the frame 4 and/or the guide 10, in position.

The third step can include, for example after the inserting, the locking of the lockable cutting window 3, for example in position. Such locking can be obtained by activating, for example turning, the window locking actuator 4i, for example located at the back of the frame 4.

The third step can include, for example after the inserting and/or the locking, the removal of the feeler 2.

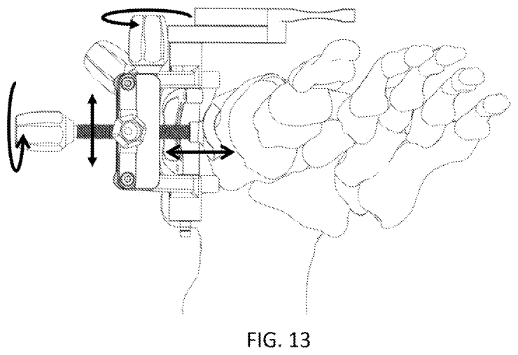

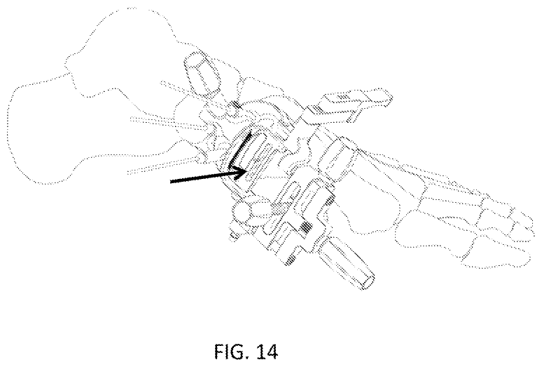

The method, for example the first part or series, can include, for example after the first step and/or second step and/or third step, a fourth step (see FIGS. 13 and 14).

The fourth step can include, performing a first cut of the second bone fragment, for example through the cutting window 3, for example through the cutting opening 3b of the cutting window 3, using the cutting tool. The cutting tool can be the percutaneous burr, for example a Ø2.2 mm percutaneous burr.

The fourth step can include, for example after or before performing the first cut, performing a second cut of the first bone fragment, for example through the cutting window 3, for example through the cutting opening 3b of the cutting window 3, using the cutting tool. The cutting tool can be the percutaneous burr, for example a Ø2.2 mm percutaneous burr.

Alternatively, performing the second cut can be performed after the sixth step as described hereunder, for example before the seventh step as described hereunder, so that the first bone fragment is cut after the angle between the first bone and the neighboring bone has been reduced.

Alternatively, performing the second cut can be performed after the eighth step as described hereunder, for example before the ninth step as described hereunder, so that the first bone fragment is cut after the angle between the first bone and the neighboring bone has been reduced and/or after the elevation/lowering of the first bone head has been performed and/or after the first bone fragment rotation around its axis has been performed. In that case, the hereunder described moving away of the cutting tool is performed after the second cut is performed.

Alternatively, the second cut is performed a first time, as described hereabove, during the first part, and is repeated during the second part, after the sixth step as described hereunder, for example before the seventh step as described hereunder. This allows for a correction of the second cut which is performed two times, before and after the angle between the first bone and the neighboring bone has been reduced.

Alternatively, the second cut is performed a first time, as described hereabove, during the first part, and is repeated during the second part, after the eighth step as described hereunder, for example before the ninth step as described hereunder. In that case, the hereunder described moving away of the cutting tool is performed after the second cut is performed. This allows for a correction of the second cut which is performed two times, before and after the angle between the first bone and the neighboring bone has been reduced and/or after the elevation/lowering has been performed and/or after the first bone fragment rotation around its axis has been performed.

The method can include a second part, for example a second series of steps, for performing correction(s), for example of at least one of the two bone fragments, for example of the relative position of the two bone fragments, for example of the position of the first bone fragment and/or of the second bone fragment, for example performed after the first part.

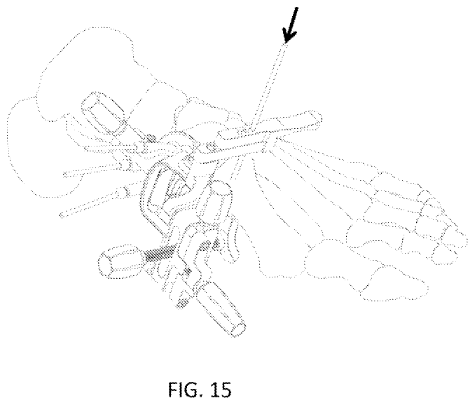

The method, for example the second part or series, can include a fifth step of positioning a rotation unit attaching pin (FIG. 15). The rotation guide 6, for example metatarsal rotation guide 6, can be used. The rotation unit attaching pin is positioned in one of the attaching pin openings 6e, for example of the cap 62, and so as to be attached to the first bone fragment. The fifth step can be implemented after the first and/or second and/or third and/or fourth step.

The positioned rotation unit attaching pin can act as a pivot point for the first bone fragment, for example for the metatarsal during its correction. Alternatively or in combination, the positioned rotation unit attaching pin, for example when operated by hand, allows the first bone fragment, for example the metatarsal, to rotate around its axis for supination and alignment, for example with the sesamoids.

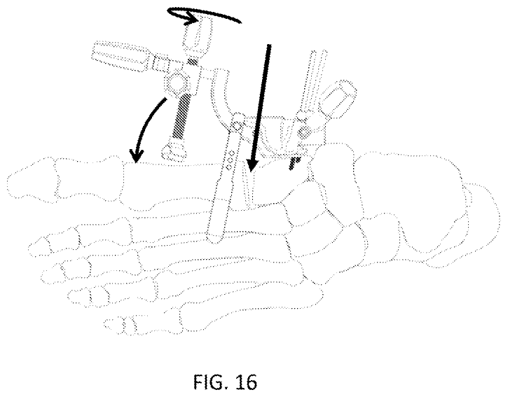

The method, for example the second part or series, can include a sixth step of reducing the angle of the first bone with a neighboring bone, for example the intermetatarsal angle (FIG. 16). The sixth step can be implemented after the first and/or second and/or third and/or fourth and/or fifth step. The reducing can be performed by using the reduction actuator 5a, for example while using the cutting tool, for example the percutaneous burr, to correct the cutting angle at the base of the first bone, for example of the metatarsal. The reduction can be performed to an angular correction deemed correct, for example by operator.

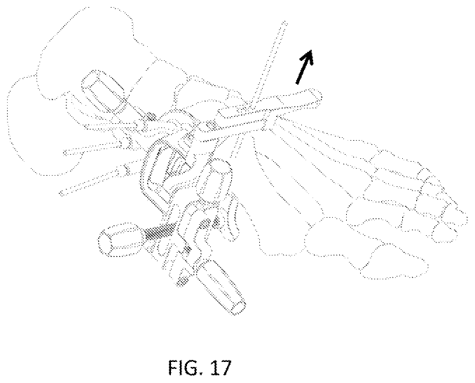

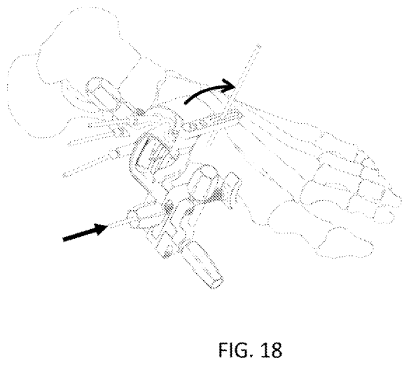

The method, for example the second part or series, can include a seventh step (FIGS. 17 and 18). The seventh step can be implemented after the first and/or second and/or third and/or fourth and/or fifth and/or sixth step.

The seventh step can include, for example after the reducing, removing the cap 62 from base 61 the rotation guide 6, for example in order to use the rotation guide attaching pin to align the first bone, for example the metatarsal, for example above the sesamoids, by supinating the first bone, for example a few degrees.

The seventh step can include, for example after the removing of the cap 62, for example by using the rotation guide attaching pin, aligning the first bone, for example the metatarsal, for example above the sesamoids, by supinating the first bone, for example a few degrees.

The seventh step can include, once the desired correction/alignment is achieved, locking the position of the first bone, for example with a locking pin to be inserted through a shaft of the reduction actuator 5a, for example medially under the first bone head, for exemple under the metatarsal head.

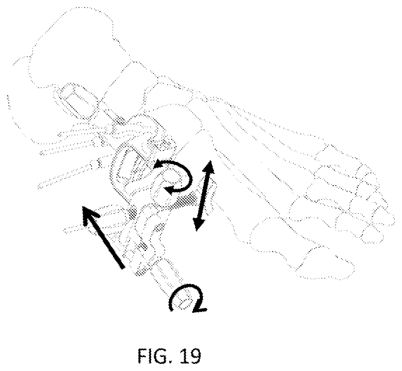

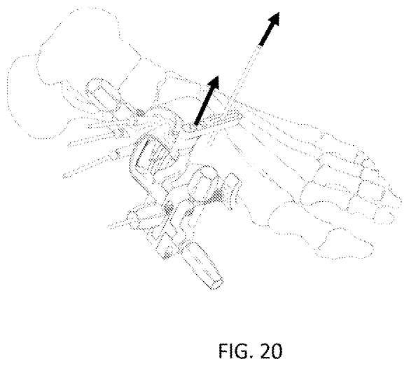

The method, for example the second part or series, can include an eighth step (FIGS. 19 and 20). The eighth step can be implemented after the first and/or second and/or third and/or fourth and/or fifth and/or sixth and/or seventh step.

The eighth step can include, at this stage, moving away the cutting tool, for example from a cutting area.

The eighth step can include, for example after the moving away, removing the rotation unit attaching pin, which forms an upper pin, and/or the rotation guide 6, for example the base 61, for example in order to finish the correction by adjusting the elevation/lowering of the first bone head, for example of the metatarsal head, with the elevation/lowering actuator 5b and then compressing the facing surfaces of the first bone fragment and of the second bone fragment, with the compression actuator 5c.

The eighth step can include, for example after the removing, adjusting the elevation/lowering of the first bone head, for example of the metatarsal head. The adjusting can be performed by the elevation/lowering actuator 5b.

The eighth step can include, for example after the removing and/or the adjusting, compressing said surfaces, for example with the compression actuator 5c.

The method can include a third part, for example a third series of steps, for aiming and implant(s) insertion, for example performed after the first part and/or second part.

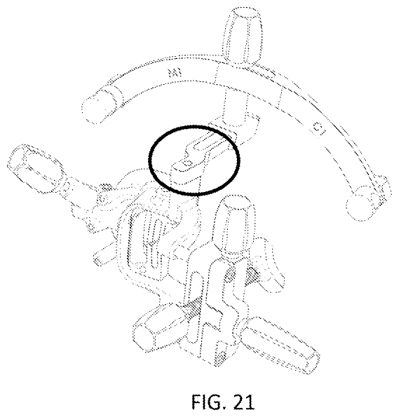

The method, for example the third part or series, can include a ninth step (FIG. 21). The ninth step include first positioning the aiming unit 7 on one of the lateral junction(s) 4b of the frame 4.

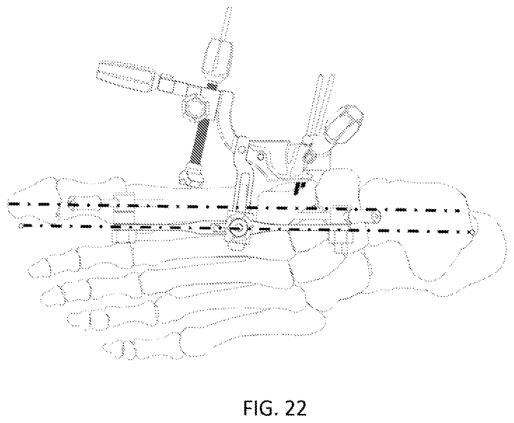

The method, for example the third part or series, can include a tenth step (FIG. 22). The tenth step is for example performed after the ninth step.

The tenth step includes aligning the aiming unit 7 with the first bone, for example parallel to the axis of the first bone, for example the metatarsal. The aligning can result in centering the barrel(s) 7g and/or 7h on the first bone. The aligning can be performed to have the best possible implant, for example screw, position using the sliding arm 7a and its pivot and/or slide 7b.

The tenth step can include, for example after the aligning, finding the desired angle for the implants, for example for the guiding pins and/or screws, for example by sliding and locking the position using the slide of the aiming arc 7c, for example on the upper side of the aiming arc 7c.

The tenth step can include, for example after the finding, locking the found position with the locking actuator 7d.

The method, for example the third part or series, can include an eleventh step. The eleventh step is for example performed after the ninth and/or tenth step. The eleventh includes inserting the barrels 7g and/or 7h, for example in the places provided, for example by respecting the indications left foot/right foot.

The method, for example the third part or series, can include a twelfth step. The twelfth step is for example performed after the ninth and/or tenth and/or eleventh step. The aiming unit 7 is now ready to receive the pins and/or implants, for example the screws.

The twelfth step can include inserting one or several guiding pins, for example the first and second guiding pins. The twelfth step can include removing the barrels 7g and/or 7h. The twelfth step can then include measuring the length of the desired implant, for example screw, for example with a graduated ruler of the system, and then using a drill to prepare the implant, for example screw, housing(s). Such step is performed without drill down to the second cortex of the bone.

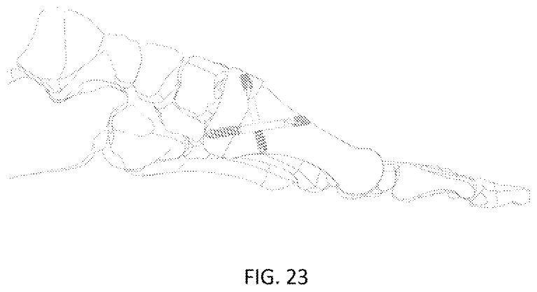

The method, for example the third part or series, can include a thirteenth step (FIG. 23). The thirteenth step is for example performed after the ninth and/or tenth and/or eleventh and/or twelfth step. The thirteenth step includes inserting a first implant, for example screw, for example of the size measured earlier, in the corresponding housing. The twelfth step can then be repeated for the second implant, for example screw. The fusion is thus completed and the system and/or guide can be removed.

The surgical method can thus include a relying on the use of the cutting tool, for example the percutaneous burr, by making the preparation phase much less invasive than with current techniques based on the oscillating saw, by offering the guide that allows cuts to be made on the cuneo-metatarsal joint, adjustable to the different corrections to be applied, whatever the anatomy.

The surgical method can include using the guide 10 to stabilize the joint in the desired final position by means of the actuators, leading to the movements necessary for the complete correction. The movements can include the reduction of an angle, for example the angle between the first metatarsal bone and the second metatarsal bone, and/or the supination of the metatarsal bone to align with the sesamoids, and/or the elevation of the metatarsal bone to maintain a good distribution of support/compression on the cuneiform.

The surgical method can include using the aiming unit 7 as an additional aimer to insert the pins for screw mounting at the desired angle and position, for example based on a crossed construct.

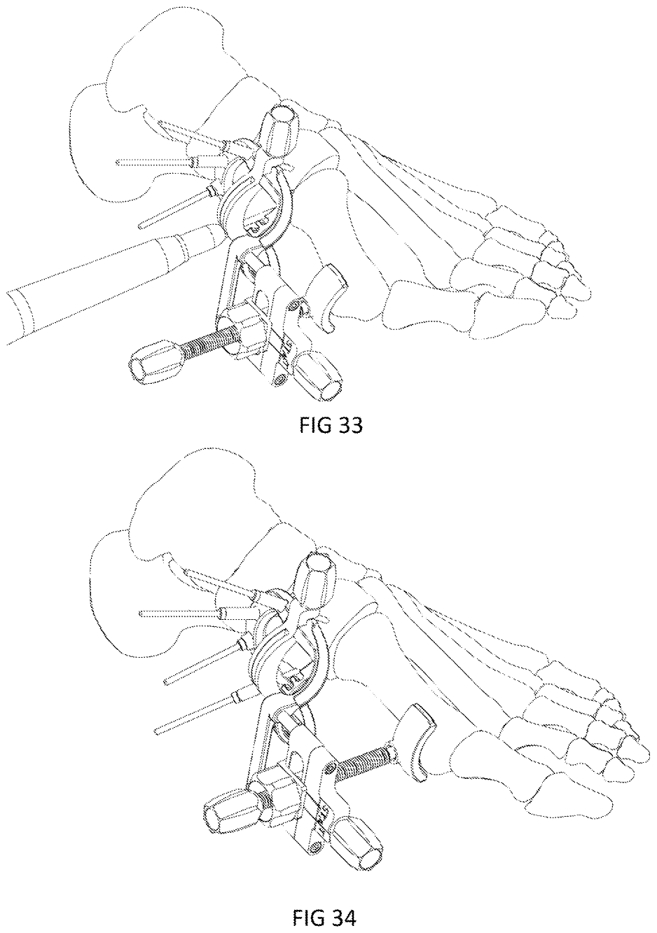

### With reference to FIGS. 33 to 43, the surgical method can include the first part or series.

The method can include the first step including creating an incision; after the creating, at least partially inserting the feeler 2.

The method can include, for example after the first step, the second step of positioning the frame, for example with the cutting window 3.

The method can include, for example after the first step and/or second step, the third step, including; the inserting one or a plurality of the frame attaching pin(s), for example four frame attaching pins; and/or after the inserting, the removal of the feeler 2.

The method, for example the first part or series, can include, for example after the first step and/or second step and/or third step, the fourth step of performing a first cut during the first part of the second bone fragment (FIG. 33).

The method can include the second part.

The method can include the sixth step of reducing the angle of the first bone with a neighboring bone, for example the intermetatarsal angle (FIG. 34). The sixth step can be implemented after the first and/or second and/or third and/or before the fourth. The reducing can be performed by using the reduction actuator 5a, to correct the cutting angle at the base of the first bone, for example of the metatarsal. The reduction can be performed to an angular correction deemed correct, for example by operator.

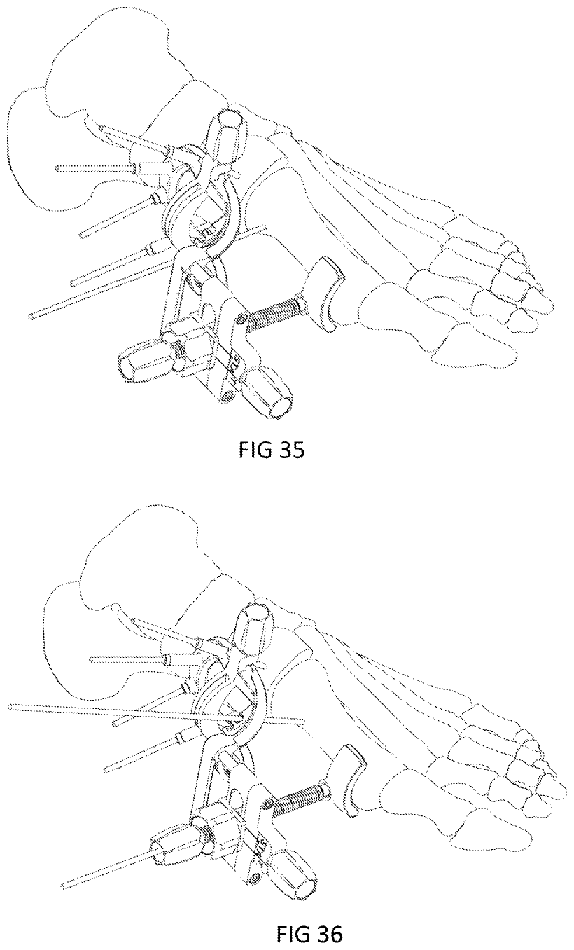

The method can include, for example after the sixth step, the fifth step of positioning a rotation unit attaching pin (FIG. 35).

The method can include, for example after the fifth step, the seventh step. The seventh step can include, by using the rotation unit attaching pin, aligning the first bone and/or; and/or once the desired correction/alignment is achieved, locking the position of the first bone, for example with a locking pin to be inserted through a shaft of the reduction actuator 5a (FIG. 36).

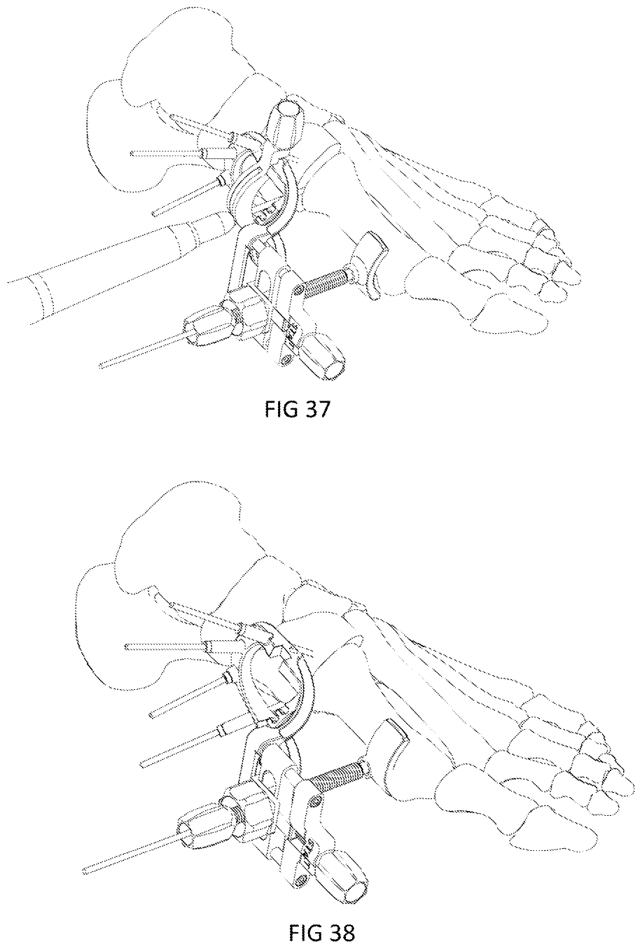

The method can include, performing the second cut, for example after the seventh step, for example before the eight step (see FIG. 37). Alternatively, the second cut can be performed after the sixth step, for example before the fifth step, and/or after the fifth step, for example before the seventh step.

The method can include the eighth step, including for example the adjusting the elevation/lowering of the first bone head, for example of the metatarsal head, and/or the compressing (FIG. 38).

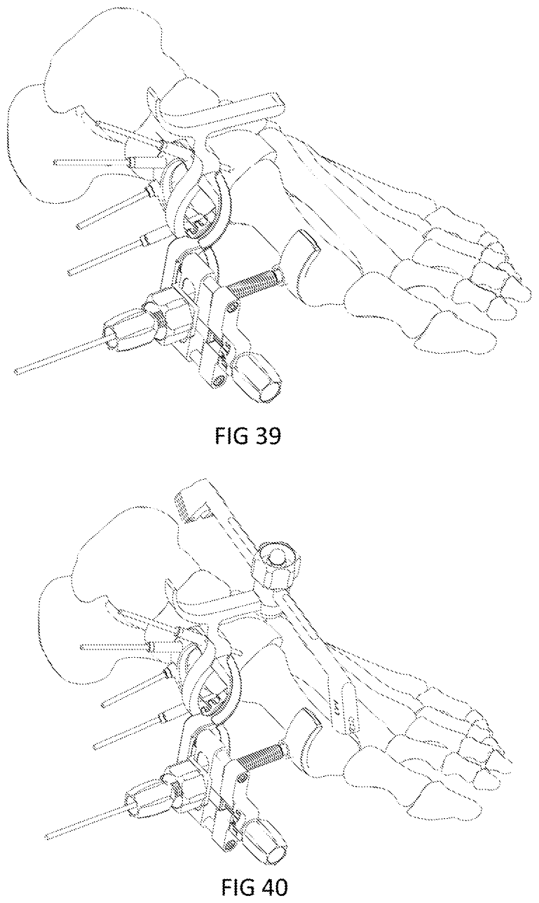

The method can include the ninth step, including the first positioning the aiming unit 7 on the window 3, for instance by positioning support subunit 71 (FIG. 39) before attaching the aiming subunit 8 thereon (FIG. 40).

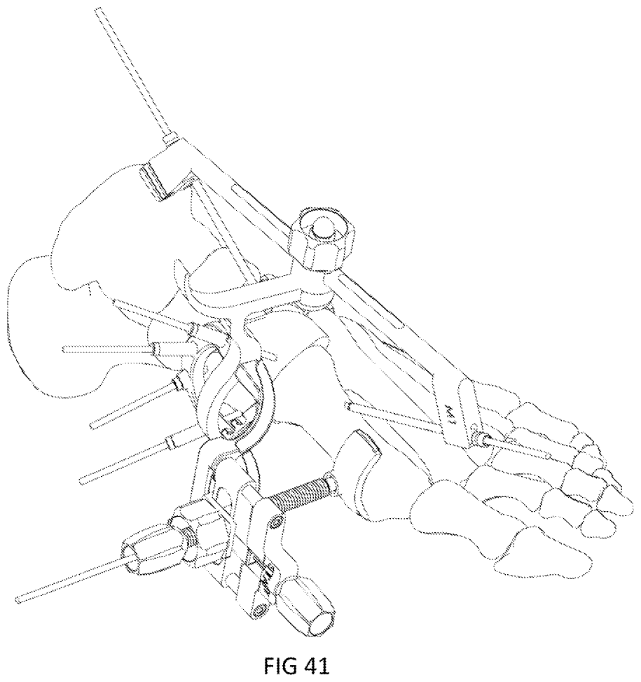

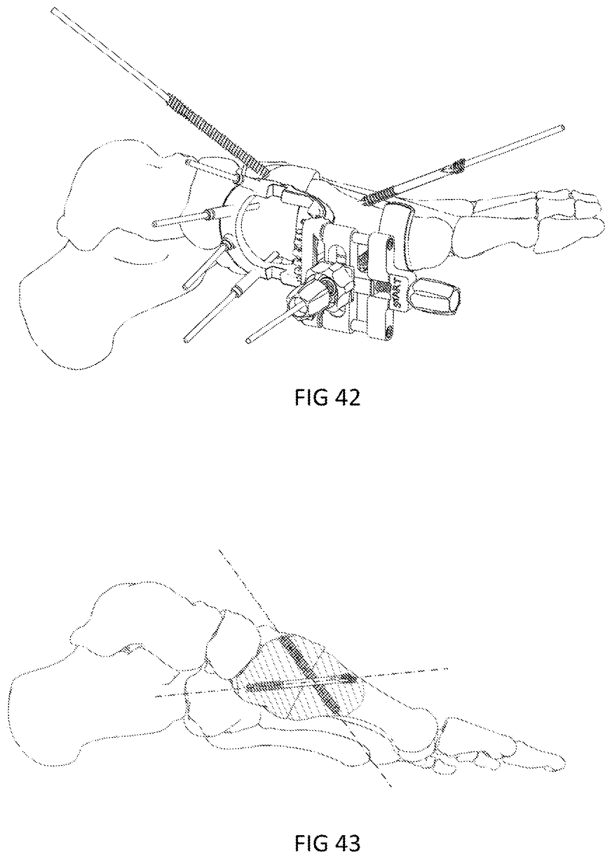

The method can include the tenth and/or eleven step(s) (FIG. 41-42).

The method can include the twelfth step(s) and/or thirteenth step (FIG. 43).

1. An orthopedic surgical guide for a surgical operation to fuse two bone fragments, wherein the guide includes:
a frame configured to be removably attached to at least one of the two bone fragments, and
a cutting window.

2. The guide according to claim 1, wherein the window is configured for allowing the passing through of at least a part of a cutting tool for cutting at least one of the two bone fragments.

3. The guide according to claim 1, wherein the window is configured to allow the insertion of at least a part of a feeler by relative displacement of the window with respect to the feeler so that the feeler guides the frame.

4. The guide according to claim 1, wherein the frame is configured to be removably attached to a single of the bone fragments to be fused.

5. The guide according to claim 1, wherein the guide further includes a correction unit.

6. The guide according to claim 1, wherein the frame and correction unit are configured so that the correction unit can be removably attached to the frame.

7. The guide according to claim 5, wherein the correction unit includes a paddle configured to be in contact with one of the two bone fragments.

8. The guide according to claim 1, wherein the guide further includes a rotation unit.

9. The guide according to claim 1, wherein the frame and rotation unit are configured so that the rotation unit can be removably attached to the frame.

10. The guide according to claim 8, wherein the rotation unit has at least one pin opening, each being configured to allow passage of a pin.

11. The guide according to claim 1, wherein the guide is configured to allow the displacement of one of the two bone fragments relative to the other of the two bone fragments, so as to allow at least two degrees in freedom.

12. The guide according to claim 1, wherein the guide further includes an aiming unit.

13. The guide according to claim 1, wherein the frame and aiming unit are configured so that the aiming unit can be removably attached to the frame.

14. The guide according to claim 12, wherein the aiming unit includes at least one slide.

15. The guide according to claim 12, wherein the aiming unit includes a first arm and a second arm, the first arm including a first housing configured for receiving a first barrel.

16. The guide according to claim 15, wherein the guide further comprises the first barrel and a second barrel, such that, when the first housing receives the first barrel, the first barrel being configured to receive a first guiding pin and the second barrel being configured to receive a second guiding pin, so as to position the first guiding pin and the second guiding pin with respect to at least one of the bone fragments,
wherein the first guiding pin being configured, when positioned, to guide a first orthopedic implant, and
wherein the second guiding pin being configured, when positioned, to guide a second orthopedic implant.

17. An orthopedic surgical guide system for a surgical operation to fuse two bone fragments, wherein the system includes:
a guide according to claim 16, and
the first guiding pin and the second guiding pin,
the first guiding pin being configured, when positioned, to guide a first orthopedic implant, and the second guiding pin being configured, when positioned, to guide a second orthopedic implant.

18. The system according to claim 17, further including the first orthopedic implant and the second orthopedic implant, wherein the first orthopedic implant is a screw and the second orthopedic implant is a screw.

19. The system according to claim 17, further including a feeler to be inserted between the two bone fragments, the feeler being configured to cooperate with the guide.

20. The system according to claim 19, wherein the window has a guiding opening for inserting at least a part of a feeler through the guiding opening by relative displacement of the window with respect to the feeler so that the feeler guides the frame.

21. The system according to claim 20, wherein the feeler has a rib adapted to cooperate with the guiding opening of the window of the guide.

22. The guide according to claim 19, wherein the feeler has a first tapered end.

23. An orthopedic surgical guide system for a surgical operation to fuse two bone fragments, wherein the system includes:
a guide according to claim 1, and
a feeler to be inserted between the two bone fragments, the feeler being configured to cooperate with the guide.

24. The system according to claim 23, wherein the window has a guiding opening for inserting at least a part of a feeler through the guiding opening by relative displacement of the window with respect to the feeler so that the feeler guides the frame.

25. The system according to claim 24, wherein the feeler has a rib adapted to cooperate with the guiding opening of the window of the guide.

26. The system according to claim 23, wherein the feeler has a first tapered end.

27. A feeler for use in a surgical operation to fuse two bone fragments, the feeler being configured to cooperate with an orthopedic surgical guide for the surgical operation to fuse two bone fragments.

28. The feeler according to claim 27, wherein the feeler has a first tapered end.

29. The feeler according to claim 27, wherein the feeler has a rib adapted to cooperate with a guiding opening of a window of the guide.

30. A surgical method to fuse two bone fragments, the method including:
providing an orthopedic surgical guide having (i) a frame configured to be removably attached to at least one of the two bone fragments and (ii) a cutting window,
performing cut(s) on the first and/or second bone fragment(s), and
performing correction(s) of the position of at least one of the two bone fragments.
